(19) United States
(12) Patent Application Publication
(10) Pub. No.: US 2004/0038965 A1
(43) Pub. Date: Feb. 26, 2004

(54) Protease inhibitors

(76) Inventors: Robert W. Marquis, Jr., King of Prussia, PA (US); Daniel F. Veber, King of Prussia, PA (US); Dennis S. Yamashita, King of Prussia, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/432,425

(22) PCT Filed: Oct. 9, 2001
(86) PCT No.: PCT/US01/42647

(51) Int. Cl. A61K 31/55; C 07D 4 3/02; C07D 223/08

(52) U.S. Cl. 514/217.04; 514/217.11; 540/597; 540/604

(57) ABSTRACT

The present invention provides C₃-C₆ 1-amino-1-acyl cycloalkane-substituted 4-amino-azepan-3-one protease inhibitors and pharmaceutically acceptable salts, hydrates and solvates thereof which inhibit proteases, including cathepsin K, pharmaceutical compositions of such compounds, novel intermediates of such compounds, and methods for treating diseases of excessive bone loss or cartilage or matrix degradation, including osteoporosis; gingival disease including gingivitis and periodontitis; arthritis, more specifically, osteoarthritis and rheumatoid arthritis; Paget's disease; hypercalcemia of malignancy; and metabolic bone disease, comprising inhibiting said bone loss or excessive cartilage or matrix degradation by administering to a patient in need thereof a compound of the present invention.

This application claims the benefit of U.S. Provisional Application No. 60/252,508, filed Nov. 22, 2000.

### FIELD OF THE INVENION

This invention relates in general to C₃-C₆ 1-amino-1-acyl cycloalkane-substituted 4-amino-azepan-3-one protease inhibitors, particularly such inhibitors of cysteine and serine proteases, more particularly compounds which inhibit cysteine proteases, even more particularly compounds which inhibit cysteine proteases of the papain superfamily, yet more particularly compounds which inhibit cysteine proteases of the cathepsin family, most particularly compounds which inhibit cathepsin K. Such compounds are particularly useful for treating diseases in which cysteine proteases are implicated, especially diseases of excessive bone or cartilage loss, e.g., osteoporosis, periodontitis, and arthritis.

### BACKGROUND OF THE INVENTION

Cathepsins are a family of enzymes which are part of the papain superfamily of cysteine proteases. Cathepsins B, H, L, N and S have been described in the literature. Recently, cathepsin K polypeptide and the cDNA encoding such polypeptide were disclosed in U.S. Pat. No. 5,501,969 (called cathepsin 0 therein). Cathepsin K has been recently expressed, purified, and characterized. Bossard, M. J., et al., (1996) J. Biol. Chem. 271, 12517-12524; Drake, F. H., et al., (1996) J. Biol. Chem. 271, 12511-12516; Bromme, D., et al., (1996) J. Biol. Chem. 271, 2126-2132.

Cathepsin K has been variously denoted as cathepsin O or cathepsin O2 in the literature. The designation cathepsin K is considered to be the more appropriate one.

Cathepsins function in the normal physiological process of protein degradation in animals, including humans, e.g., in the degradation of connective tissue. However, elevated levels of these enzymes in the body can result in pathological conditions leading to disease. Thus, cathepsins have been implicated as causative agents in various disease states, including but not limited to, infections by pneumocystis carinii, trypsanoma cruzi, trypsanoma brucei brucei, and Crithidia fusiculata; as well as in schistosomiasis, malaria, tumor metastasis, metachromatic leukodystrophy, muscular dystrophy, amytrophy, and the like. See International Publication Number WO 94/04172, published on Mar. 3, 1994, and references cited therein. See also European Patent Application EP 0 603 873 A1, and references cited therein. Two bacterial cysteine proteases from P. gingivallis, called gingipains, have been implicated in the pathogenesis of gingivitis. Potempa, J., et al. (1994) Perspectives in Drug Discovery and Design, 2, 445-458.

Cathepsin K is believed to play a causative role in diseases of excessive bone or cartilage loss. Bone is composed of a protein matrix in which spindle- or plate-shaped crystals of hydroxyapatite are incorporated. Type I collagen represents the major structural protein of bone comprising approximately 90% of the protein matrix. The remaining 10% of matrix is composed of a number of non-collagenous proteins, including osteocalcin, proteoglycans, osteopontin, osteonectin, thrombospondin, fibronectin, and bone sialoprotein. Skeletal bone undergoes remodelling at discrete foci throughout life. These foci, or remodelling units, undergo a cycle consisting of a bone resorption phase followed by a phase of bone replacement.

Bone resorption is carried out by osteoclasts, which are multinuclear cells of hematopoietic lineage. The osteoclasts adhere to the bone surface and form a tight sealing zone, followed by extensive membrane ruffling on their apical (i.e., resorbing) surface. This creates an enclosed extracellular compartment on the bone surface that is acidified by proton pumps in the ruffled membrane, and into which the osteoclast secretes proteolytic enzymes. The low pH of the compartment dissolves hydroxyapatite crystals at the bone surface, while the proteolytic enzymes digest the protein matrix. In this way, a resorption lacuna, or pit, is formed. At the end of this phase of the cycle, osteoblasts lay down a new protein matrix that is subsequently mineralized. In several disease states, such as osteoporosis and Paget's disease, the normal balance between bone resorption and formation is disrupted, and there is a net loss of bone at each cycle. Ultimately, this leads to weakening of the bone and may result in increased fracture risk with minimal trauma.

Several published studies have demonstrated that inhibitors of cysteine proteases are effective at inhibiting osteoclast-mediated bone resorption, and indicate an essential role for a cysteine proteases in bone resorption. For example, Delaisse, et al., Biochem. J., 1980, 192, 365, disclose a series of protease inhibitors in a mouse bone organ culture system and suggest that inhibitors of cysteine proteases (e.g., leupeptin, Z-Phe-Ala-CHN₂) prevent bone resorption, while serine protease inhibitors were ineffective. Delaisse, et al., Biochem Biophys. Res. Commun., 1984, 125, 441, disclose that E-64 and leupeptin are also effective at preventing bone resorption in vivo, as measured by acute changes in serum calcium in rats on calcium deficient diets. Lerner, et al., J. Bone Min. Res., 1992, 7,433, disclose that cystatin, an endogenous cysteine protease inhibitor, inhibits PTH stimulated bone resorption in mouse calvariae. Other studies, such as by Delaisse, et al., Bone, 1987, 8, 305, Hill, et al., J. Cell. Biochem., 1994, 56, 118, and Everts, et al., J. Cell. Physiol., 1992, 150, 221, also report a correlation between inhibition of cysteine protease activity and bone resorption. Tezuka, et al., J. Biol. Chem., 1994, 269, 1106, Inaoka, et al., Biochem. Biophys. Res. Commun., 1995, 206, 89 and Shi, et al., FEBS Lett., 1995, 357, 129 disclose that under normal conditions cathepsin K, a cysteine protease, is abundantly expressed in osteoclasts and may be the major cysteine protease present in these cells.

The abundant selective expression of cathepsin K in osteoclasts strongly suggests that this enzyme is essential for bone resorption. Thus, selective inhibition of cathepsin K may provide an effective treatment for diseases of excessive bone loss, including, but not limited to, osteoporosis, gingival diseases such as gingivitis and periodontitis, Paget's disease, hypercalcemia of malignancy, and metabolic bone disease. Cathepsin K levels have also been demonstrated to be elevated in chondroclasts of osteoarthritic synovium. Thus, selective inhibition of cathepsin K may also be useful for treating diseases of excessive cartilage or matrix degradation, including, but not limited to, osteoarthritis and rheumatoid arthritis. Metastatic neoplastic cells also typically express high levels of proteolytic enzymes that degrade the surrounding matrix. Thus, selective inhibition of cathepsin K may also be useful for treating certain neoplastic diseases.

Several cysteine protease inhibitors are known. Palmer, (1995) J. Med. Chem., 38, 3193, disclose certain vinyl sulfones which irreversibly inhibit cysteine proteases, such as the cathepsins B, L, S, O2 and cruzain. Other classes of compounds, such as aldehydes, nitriles, o-ketocarbonyl compounds, halomethyl ketones, diazomethyl ketones, (acyloxy)methyl ketones, ketomethylsulfonium salts and epoxy succinyl compounds have also been reported to inhibit cysteine proteases. See Palmer, id, and references cited therein.

U.S. Pat. No. 4,518,528 discloses peptidyl fluoromethyl ketones as irreversible inhibitors of cysteine protease. Published International Patent Application No. WO 94/04172, and European Patent Application Nos. EP 0 525 420 A1, EP 0 603 873 A1, and EP 0 611 756 A2 describe alkoxymethyl and mercaptomethyl ketones which inhibit the cysteine proteases cathepsins B, H and L. International Patent Application No. PCT/US94/08868 and and European Patent Application No. EP 0 623 592 A1 describe alkoxymethyl and mercaptomethyl ketones which inhibit the cysteine protease IL-1βconvertase. Alkoxymethyl and mercaptomethyl ketones have also been described as inhibitors of the serine protease kininogenase (International Patent Application No. PCT/GB91/01479).

Azapeptides which are designed to deliver the azamino acid to the active site of serine proteases, and which possess a good leaving group, are disclosed by Elmore et al., Biochem J., 1968, 107, 103, Garker et al., Biochem. J., 1974, 139, 555, Gray et al., Tetrahedron, 1977, 33, 837, Gupton et al., J. Biol. Chem., 1984, 259, 4279, Powers et al., J. Biol. Chem., 1984,259,4288, and are known to inhibit serine proteases. In addition, J. Med. Chem, 1992, 35, 4279, discloses certain azapeptide esters as cysteine protease inhibitors.

Antipain and leupeptin are described as reversible inhibitors of cysteine protease in McConnell et al., J. Med. Chem., 33, 86; and also have been disclosed as inhibitors of serine protease in Umezawa et al., 45 Meth. Enzymol. 678. E64 and its synthetic analogs are also well-known cysteine protease inhibitors (Barrett, Biochem. J., 201, 189, and Grinde, Biochem. Biophys. Acta, 701, 328).

1,3-diamido-propanones have been described as analgesic agents in U.S. Pat. Nos. 4,749,792 and 4,638,010.

EP 1 008 592 A2 describes cyclic amide derivatives which inhibit cathepsin K.

Thus, a structurally diverse variety of protease inhibitors have been identified. However, these known inhibitors are not considered suitable for use as therapeutic agents in animals, especially humans, because they suffer from various shortcomings. These shortcomings include lack of selectivity, cytotoxicity, poor solubility, and overly rapid plasma clearance. A need therefore exists for methods of treating diseases caused by pathological levels of proteases, particularly cysteine proteases, more particularly cathepsins, most particularly cathepsin K, and for novel inhibitor compounds useful in such methods.

We have now discovered a novel class of C₃-C₆ 1-amino-1-acyl cycloalkane-substituted 4-amino-azepan-3-one compounds which are protease inhibitors, most particularly of cathepsin K.

### SUMMARY OF THE INVENTION

An object of the present invention is to provide C₃-C₆ 1-amino-1-acyl cycloalkane-substituted 4-amino-azepan-3-one carbonyl protease inhibitors, particularly such inhibitors of cysteine and serine proteases, more particularly such compounds which inhibit cysteine proteases, even more particularly such compounds which inhibit cysteine proteases of the papain superfamily, yet more particularly such compounds which inhibit cysteine proteases of the cathepsin family, most particularly such compounds which inhibit cathepsin K, and which are useful for treating diseases which may be therapeutically modified by altering the activity of such proteases.

Accordingly, in the first aspect, this invention provides a compound according to Formula I.

In another aspect, this invention provides a pharmaceutical composition comprising a compound according to Formula I and a pharmaceutically acceptable carrier, diluent or excipient.

In yet another aspect, this invention provides intermediates useful in the preparation of the compounds of Formula I.

In still another aspect, this invention provides a method of treating diseases in which the disease pathology may be therapeutically modified by inhibiting proteases, particularly cysteine and serine proteases, more particularly cysteine proteases, even more particularly cysteine proteases of the papain superfamily, yet more particularly cysteine proteases of the cathepsin family, most particularly cathepsin K.

In a particular aspect, the compounds of this invention are especially useful for treating diseases characterized by bone loss, such as osteoporosis and gingival diseases, such as gingivitis and periodontitis, or by excessive cartilage or matrix degradation, such as osteoarthritis and rheumatoid arthritis.

### DETAILED DESCRIPTION OF THE INVENTION

The present invention provides compounds of Formula I:

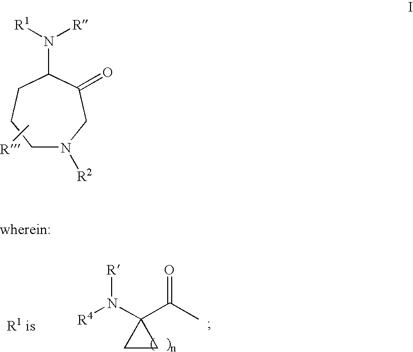

R² is selected from the group consisting of: H, C₁₋₆alkyl, C₃₋₆cycloalkyl-C₀₋₆alkyl, Ar—C₀₋₆alkyl, Het-C₀₋₆alkyl, R⁹C(O)—, R⁹C(S)—, R⁹SO₂—, R⁹OC(O)—, R⁹R¹¹NC(O)—, R⁹R¹¹NC(S)—, R⁹(R¹¹)NSO₂—,

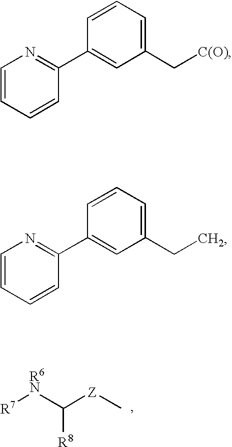

and R⁹SO₂R¹¹NC(O)—;

R⁴ is selected from the group consisting of: H, C₁₋₆alkyl, C₃₋₆cycloalkyl-C₀₋₆alkyl, Ar—C₀₋₆alkyl, Het-C₀₋₆alkyl, R⁵C(O)—, R⁵C(S)—, R⁵SO₂—, R⁵OC(O)—, R⁵R¹²NC(O)—, and R⁵R¹²NC(S)—;

R⁵ is selected from the group consisting of: H, C₁₋₆alkyl, C₂₋₆alkenyl, C₂₋₆alkynyl, C₃₋₆cycloalkyl-C₀₋₆alkyl, C₂₋₆alkanonyl, Ar—C₀₋₆alkyl and Het-C₀₋₆alkyl;

R⁶ is selected from the group consisting of: H, C₁₋₆alkyl, Ar—C₀₋₆alkyl, and Het-C₀₋₆alkyl;

R⁷ is selected from the group consisting of: H, C₁₋₆alkyl, C₃₋₆cycloalkyl-C₀₋₆alkyl, Ar—C₀₋₆alkyl, Het-C₀₋₆alkyl, R¹⁰C(O)—, R¹⁰C(S)—, R¹⁰SO₂—, R¹⁰OC(O)—, R¹⁰R¹³NC(O)—, and R¹⁰R¹³NC(S)—;

R⁸ is selected from the group consisting of: H, C₁₋₆alkyl, C₂₋₆alkenyl, C₂₋₆alkynyl, HetC₀₋₆alkyl and ArC₀₋₆alkyl;

R⁹ is selected from the group consisting of: C₁₋₆alkyl, C₃₋₆cycloalkyl-C₀₋₆alkyl, Ar—C₀₋₆alkyl, —Ar—COOH, and Het-C₀₋₆alkyl;

R¹⁰ is selected from the group consisting of: C₁₋₆alkyl, C₃₋₆cycloalkyl-C₀₋₆alkyl, Ar—C₀₋₆alkyl and Het-C₀₋₆alkyl;

R¹¹ is selected from the group consisting of: H, C₁₋₆alkyl, Ar—C₀₋₆alkyl, and Het-C₀₋₆alkyl;

R¹² is selected from the group consisting of: H, C₁₋₆alkyl, Ar—C₀₋₆alkyl, and Het-C₀₋₆alkyl;

R¹³ is selected from the group consisting of: H, C₁₋₆alkyl, Ar—C₀₋₆alkyl, and Het-C₀₋₆alkyl;

R′ is selected from the group consisting of: H, C₁₋₆alkyl, Ar—C₀₋₆alkyl, and Het-C₀₋₆alkyl;

R″ is selected from the group consisting of: H, C₁₋₆alkyl, Ar—C₀₋₆alkyl, and Het-C₀₋₆alkyl;

R′″ is selected from the group consisting of: H, C₁₋₆alkyl, C₃₋₆cycloalkyl-C₀₋₆alkyl, Ar—C₀₋₆alkyl, and Het-C₀₋₆alkyl;

Z is selected from the group consisting of: C(O) and CH₂;

n is an integer from 1 to 5;

and pharmaceutically acceptable salts, hydrates and solvates thereof.

In compounds of Formula I, n is preferably 4, to provide 1-amino-1-acyl cyclohexane compounds. The cycloalkyl ring may be unsubstituted or substituted with one or more of C₁₋₆alkyl, C₃₋₆cycloalkyl-C₀₋₆alkyl, C₂₋₆alkenyl, C₂₋₆alkynyl, HetC₀₋₆alkyl, ArC₀₋₆alkyl, or halogen.

The cycloalkyl ring is more preferably unsubstituted.

R⁴ is selected from the group consisting of: H, C₁₋₆alkyl, C₃₋₆cycloalkyl-C₀₋₆alkyl, Ar—C₀₋₆alkyl, Het-C₀₋₆alkyl, R⁵C(O)—, R⁵C(S)—, R⁵SO₂—, R⁵OC(O)—, R⁵R¹³NC(O)—, and R⁵R¹³NC(S)—.

R⁴ is preferably selected from the group consisting of: R⁵OC(O)—, R⁵C(O)— and R⁵SO₂—.

R⁴ is most preferably R⁵C(O)—.

In some embodiments, R⁴ is preferably methanesulfonyl.

R⁵ is selected from the group consisting of: H, C₁₋₆alkyl, C₂₋₆alkenyl, C₂₋₆alkynyl, C₃₋₆cycloalkyl-C₀₋₆alkyl, C₂₋₆alkanonyl, Ar—C₀₋₆alkyl or Het-C₀₋₆alkyl.

Preferably R⁵ is selected from the group consisting of: C₁₋₆alkyl, C₂₋₆alkenyl, C₂₋₆alkynyl, C₃₋₆cycloalkyl-C₀₋₆alkyl, C₂₋₆alkanonyl, Ar—C₀₋₆alkyl and Het-C₀₋₆alkyl.

More preferably, and especially when R⁴ is R⁵C(O)—, R⁵ is selected from the group consisting of:

methyl, especially halogenated methyl, more especially trifluoromethyl, especially C₁₋₆alkoxy and aryloxy substituted methyl, more especially phenoxy-methyl, 4-fluoro-enoxy-methyl, especially heterocycle substituted methyl, more especially 2-thiophenylethyl;

ethyl, especially piperidin-1-yl-ethyl;

butyl, especially aryl substituted butyl, more especially 4(4-methoxy)phenyl-butyl;

isopentyl;

cyclohexyl;

butenyl, especially aryl substituted butenyl, more especially 4,4-bis(4-ethoxyphenyl)-but-3-enyl;

acetyl;

pentanonyl, especially 4-pentanonyl;

phenyl, especially phenyl substituted with one or more halogens, more especially 3,4-dichlorophenyl and 4-fluorophenyl, especially phenyl substituted with one or more aryloxy or C₁₋₆alkoxy groups, more especially 3,4-dimethoxy-phenyl, 3-benzyloxy-4-methoxy-phenyl, especially phenyl substituted with one or more C₁₋₆alkyl sulfonyl groups, more especially 4-methanesulfonyl-phenyl;

benzyl;

naphthalenyl, especially naphthylen-2-yl;

benzo[1,3]dioxolyl, especially benzo[1,3]dioxol-5-yl;

furanyl, especially furan-2-yl, especially nitro substituted furanyl, more especially 5-nitro-furan-2-yl, more especially halogen substituted furanyl, even more especially 5-bromo-furan-2-yl, more especially aryl substituted furanyl, even more especially 5-(4-chloro-phenyl)-furan-2-yl, 5-(4-nitrophenyl)-furan-2-yl, 5-(3-trifluoromethyl-phenyl)-furan-2-yl, more especially C₁₋₆alkyl substituted furanyl, even more especially 3-methyl-furan-2-yl, 4-methyl-furan-2-yl, 2,5-dimethyl-furan-2-yl, and 2,4-dimethyl-furan-3-yl;

tetrahydrofuranyl, especially tetrahydrofuran-2-yl;

benzofuranyl, especially benzofuran-2-yl, especially C₁₋₆alkoxy substituted benzofuranyl, more especially 7-methoxy-benzofuran-2-yl, 5-methoxy-benzofuran-2-yl, 5,6-dimethoxy-benzofuran-2-yl, 5-(2-piperazin-4-carboxylic acid tert-butyl ester-ethoxy) benzofuran-2-yl, 5-(2-morpholino-4-yl-ethoxy)-benzofuran-2-yl, 5-(2-piperazin-1-yl-ethoxy)benzofuran-2-yl, 5-(2-cyclohexyl-ethoxy)-benzofuran-2-yl, 5-methoxy-3-methyl-benzofuran-2-yl, 4-methoxy-3-methyl-benzofuran-2-yl, and 6-methoxy-3-methyl-benzofuran-2-yl; especially halogen substituted benzofuranyl, more especially 5-fluoro-benzofuran-2-yl, 5,6-difluoro-benzofuran-2-yl, 5-fluoro-3-methyl-benzofuran-2-yl, 6-fluoro-3-methyl-benzofuran-2-yl; especially C₁₋₆alkyl substituted benzofuranyl, most especially 3-methyl-benzofuran-2-yl, 3,5-dimethyl-benzofuran-2-yl, and 3-ethyl-benzofuran-2-yl;

naphtho[2,1-b]-furanyl, especially naphtho[2,1-b]-furan-2-yl, alkyl substituted naphtho[2,1-b]-furanyl, especially 1-methyl-naphtho[2,1-b]-furan-2-yl;

benzo[b]thiophenyl, especially benzo[b]thiophen-2-yl; especially C₁₋₆alkoxy substituted benzo[b]thiophenyl, more especially 5,6-dimethoxy-benzo[b]thiophen-2-yl;

quinolinyl, especially quinolin-2-yl, quinolin-3-yl, quinolin-4-yl, quinolin-6-yl, and quinolin-8-yl;

quinoxalinyl, especially quinoxalin-2-yl;

1,8 naphthyridinyl, especially 1,8 naphthyridin-2-yl;

indolyl, especially indol-2-yl, indol-6-yl, indol-5-yl, especially C₁₋₆alkyl substituted indolyl, more especially N-methyl-indol-2-yl;

pyridinyl, especially pyridin-2-yl, pyridin-3-yl, pyridin-5-yl, especially C₁₋₆alkyl substituted pyridinyl, more especially 2-methyl-pyridin-5-yl, and oxy-pyridinyl, especially 1-oxy-pyridin-2-yland 1-oxy-pyridin-3-yl;

furo[3,2-b]-pyridinyl, especially furo[3,2-b]-pyridin-2-yl, C₁₋₆alkyl substituted furo[3,2-b]-pyridinyl, especially 3-methyl-ftiro[3,2-b]-pyridin-2-yl;

thiopheneyl, especially thiophene-3-yl, also thiophene-2-yl, especially C₁₋₆alkyl substituted thiopheneyl, more especially 5-methyl-thiophene-2-yland 5-methyl-thiophene-3-yl, especially halogen substituted thiopheneyl, more especially 4,5-dibromo-thiophene-2-yl;

thieno[3,2-b]thiophene, especially thieno[3,2-b]thiophene-2-yl, more especially C₁₋₆alkyl substituted thieno[3,2-b]thiophene-2-yl, more especially 5-ten-butyl-3-methyl-thieno[3,2-b]thiophene-2-yl;

isoxazolyl, especially isoxazol-4-yl, especially C₁₋₆alkyl substituted isoxazolyl, more especially 3,5-dimethyl-isoxazol-4-yl;

oxazolyl, especially oxazol-4-yl, especially aryl substituted oxazolyl, more especially 5-methyl-2-phenyl oxazolyl, especially C₁₋₆alkyl substituted oxazolyl, more especially 2-phenyl-5-trifluoromethyl-oxazol-4-yl; and

1H-benzoimidazolyl, especially 1H-benzoimidazol-5-yl.

When R⁴ is R⁵SO₂, R⁵ is preferably pyridin-2-yl or 1-oxo-pyridin-2-yl.

R′ is selected from the group consisting of: H, C₁₋₆alkyl, Ar—C₀₋₆alkyl, and Het-C₀₋₆alkyl.

Preferably R′ is selected from the group consisting of: H and naphthalen-2-yl-methyl.

Most preferably R′ is H.

R″ is selected from the group consisting of: H, C₁₋₆alkyl, Ar—C₀₋₆alkyl, and Het-C₀₋₆alkyl.

Most preferably R″ is H.

R′″ is selected from the group consisting of: H, C₁₋₆alkyl, C₃₋₆cycloalkyl-C₀₋₆alkyl, and Het-C₀₋₆alkyl.

R′″ is preferably selected from the group consisting of: H and C₁₋₆alkyl.

R′″ is more preferably selected from the group consisting of: H and methyl.

Most preferably R′″ is methyl.

In compounds of Formula I, R² is selected from the group consisting of:

H, C₁₋₆alkyl, C₃₋₆cycloalkyl-C₀₋₆alkyl, Ar—C₀₋₆alkyl, Het-C₀₋₆alkyl, R⁹C(O)—, R⁹C(S)—, R⁹SO₂—, R⁹OC(O)—, R⁹R¹¹NC(O)—, R⁹R¹¹NC(S)—, R⁹R¹¹NSO₂—,

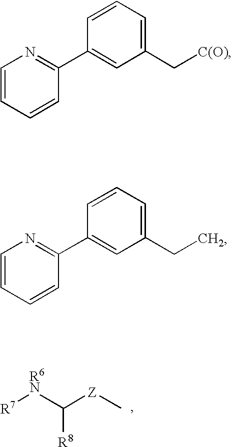

, and R⁹SO₂R¹¹NC(O)—.

More preferably R² is selected from the group consisting of: Ar—C₀₋₆alkyl, R⁹C(O)—, R⁹SO₂, R⁹R¹¹NC(O)—, and

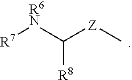

Even more preferably, R² is selected from the group consisting of: Ar—C₀₋₆alkyl, R⁹C(O)—, and R⁹SO₂.

Most preferably R² is R⁹SO₂.

In such embodiments:

R⁶ is selected from the group consisting of: H, C₁₋₆alkyl, Ar—C₀₋₆alkyl, or Het-C₀₋₆alkyl, preferably H.

R⁷ is selected from the group consisting of: H, C₁₋₆alkyl, C₃₋₆cycloalkyl-C₀₋₆alkyl, Ar—C₀₋₆alkyl, Het-C₀₋₆alkyl, R¹⁰C(O)—, R¹OC(S)—, R¹⁰SO₂—, R¹⁰OC(O)—, R¹⁰R¹⁴NC(O)—, and R¹⁰R¹⁴NC(S)—. R⁷ is preferably R¹⁰OC(O).

R⁸ is selected from the group consisting of: H, C₁₋₆alkyl, C₂₋₆alkenyl, C₂₋₆alkynyl, HetC₀₋₆alkyl and ArC₀₋₆alkyl; preferably C₁₋₆alkyl, more preferably isobutyl.

R⁹ is selected from the group consisting of: C₁₋₆alkyl, C₃₋₆cycloalkyl-C₀₋₆alkyl, Ar—C₀₋₆alkyl, —Ar—COOH, and Het-C₀₋₆alkyl.

R⁹ is preferably selected from the group consisting of: C₁₋₆alkyl, Ar—C₀₋₆alkyl, —Ar—COOH, and Het-C₀₋₆alkyl.

More preferably, R⁹ is selected from the group consisting of:

methyl;

ethyl, especially C₃₋₆cycloalkyl-C₀₋₆alkyl-substituted ethyl, more especially 2-cyclohexyl-ethyl;

propyl;

butyl, especially C₁₋₆alkylbutyl, more especially 3-methylbutyl;

tert-butyl, particularly when R² is R⁹OC(O);

isopentyl;

phenyl, especially halogen substituted phenyl, more especially 3,4-dichlorophenyl, 4-bromophenyl, 2-fluorophenyl, 3-fluorophenyl, 4-fluorophenyl, 2-chloropbenyl, 3-chlorophenyl, 4-chlorophenyl, especially C₁₋₆alkoxy phenyl, more especially 3-methoxyphenyl, 4-methoxyphenyl, 3,4-dimethoxyphenyl, especially cyanophenyl, more especially 2-cyanophenyl; especially C₁₋₆alkyl substituted phenyl, more especially 4-ethyl-phenyl, 2-methyl phenyl, 4-methyl phenyl, especially C₁₋₆alkyl sulfonyl substituted phenyl, more especially 4-methanesulfonyl phenyl, and 2-methanesulfonyl phenyl;

toluyl, especially Het-substituted toluyl, more especially 3-(pyridin-2-yl)toluyl;

naphthylene, especially naphthyl-2-ene;

benzoic acid, especially 2-benzoic acid;

benzo[1,3]dioxolyl, especially benzo[1,3]dioxol-5-yl;

benzo[1,2,5]oxadiazolyl, especially benzo[1,2,5]oxadiazol-4-yl;

pyridinyl, especially pyridin-2-yl, pyridin-3-yl, especially 1-oxy-pyridinyl, more especially 1-oxy-pyridin-2-yl, 1-oxy-pyridin-3-yl; especially C₁₋₆alkylpyridinyl, more especially 3-methyl-pyridin-2-yl, 6-methyl-pyridin-2-yl;

thiopheneyl, especially thiophene-2-yl;

thiazolyl, especially thiazol-2-yl;

1H-imidazolyl, especially 1H-imidazol-2-yl, 1H-imidazol-4-yl, more especially C₁₋₆alkyl substituted imidazolyl, even more especially 1-methyl-1H-imidazol-2-yl, 1-methyl-1H-imidazol-4-yl, and 1,2-dimethyl-1H-imidazol-4-yl;

triazolyl, especially 1H-[1,2,4]triazolyl, more especially 1H-[1,2,4]triazol-3-yl, especially C₁₋₆alkyl substituted 1H-[1,2,4]-triazolyl, more especially 5-methyl-1H-[1,2,4]triazol-3-yl; and

isoxazolyl, especially isoxazolyl, especially C₁₋₆alkyl substituted isoxazolyl, more especially 3,5-dimethyl-isoxazol-4-yl.

When R² is R⁹SO₂, R⁹ is most preferably selected from the group consisting of: pyridin-2-yl and 1-oxy-pyridin-2-yl.

When R² is R⁹SO₂R¹¹NC(O)—, R⁹ is preferably Ar—C₀₋₆alkyl, more preferably Ar, most preferably substituted phenyl such as 2-methyl phenyl, 4-methyl phenyl, 2-chloro phenyl, and 4-fluoro phenyl. R¹¹ is selected from the group consisting of: H, C₁₋₆alkyl, Ar—C₀₋₆alkyl, and Het-C₀₋₆alkyl, When R² is R⁹SO₂R¹¹NC(O)—, R¹¹ is preferably H.

When R² is R⁹C(O)—, R⁹ is preferably selected from the group consisting of C₁₋₆alkyl, C₃₋₆cycloalkyl-C₀₋₆alkyl, and Het-C₀₋₆alkyl, more preferably 1-oxy-pyridin-2-yl, cyclohexyl ethyl, and 3-methyl butyl.

When R² is Ar—C₀₋₆alkyl, R² is preferably phenyl, especially substituted phenyl, more especially halogen substituted phenyl, even more especially 2-fluorobenzyl.

When R² is C₁₋₆alkyl, R² is preferably selected from 1-propyl, 1-butyl, and 1-pentyl.

When R² is Het-C₀₋₆alkyl, Het-C₀₋₆alkyl is preferably Het-methyl, and Het in Het-methyl is preferably selected from the group consisting of:

pyridinyl, especially pyridin-2-yl, especially C₁₋₆alkylpyridinyl, more especially 6-methyl-pyridin-2-yl;

thiopheneyl, especially thiophene-2-yl, more especially thiophene-2-yl or benzo[b]thiophene-2-yl;

thiazolyl, especially thiazol-4-yl such as 1-(2-morpholin-4-yl-thiazol-4-yl), and 1-(isothiazol-3-yl);

1H-imidazolyl, especially 1H-imidazol-2-yl, 1H-imidazol-4-yl, especially C₁₋₆alkyl substituted imidazolyl, more especially 1-methyl-1H-imidazol-2-yl;

triazolyl, especially 3H-[1,2,3]triazolyl, more especially 3H-[1,2,3]triazol-4-yl, especially C₁₋₆alkyl substituted 3H-[1,2,3]triazolyl, more especially 3-phenyl-3H-[1,2,3]triazolyl-4-yl;

quinolinyl, especially quinolin-2-yl, quinolin-2-yl;

furanyl, especially furan-2-yl, especially substituted furanyl, such as 5-ethyl-furan-2-yl; and

thieno[3,2-b]thiophene, especially thieno[3,2-b]thiophene-2-yl, especially C₁₋₆alkyl substituted thieno[3,2-b]thiopheneyl, especially 3,4-dimethyl-thieno[3,2-b]thiophene-2-yl.

R² is also preferably:

H;

toluyl;

aryl substituted ethyl, especially 2-phenyl ethyl, 2-[3-(pyridin-2-yl) phenyl] ethyl.

Compounds of Formula I where R″ is H and R′″ is methyl are preferred.

More preferred are compounds of Formula I wherein:

R² is selected from the group consisting of: Ar—C₀₋₆alkyl, R⁹C(O)—, R⁹SO₂, R⁹R¹¹NC(O)—, and

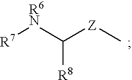

R⁴ is selected from the group consisting of: R⁵OC(O)—, R⁵C(O)— and R⁵SO₂—;

R⁵ is selected from the group consisting of: C₁₋₆alkyl, C₂₋₆alkenyl, C₂₋₆alkynyl, C₃₋₆cycloalkyl-C₀₋₆alkyl, C₂₋₆alkanonyl, Ar—C₀₋₆alkyl and Het-C₀₋₆alkyl;

R⁶ is H;

R⁷ is R¹⁰OC(O);

R⁸ is C₁₋₆alkyl;

R⁹ is selected from the group consisting of: C₁₋₆alkyl, Ar—C₀₋₆alkyl, —Ar—COOH, and Het-C₀₋₆alkyl;

R¹⁰ is selected from the group consisting of: C₁₋₆alkyl, Ar—C₀₋₆alkyl and Het-C₀₋₆alkyl;

R′ is H;

R″ is H;

R′″ is methyl; and

Z is selected from the group consisting of: C(O) and CH₂.

Even more preferred are such compounds of Formula I wherein R² is selected from the group consisting of: Ar—C₀₋₆alkyl, R⁹C(O)—, R⁹SO₂.

Yet more preferred are compounds of Formula I wherein:

R² is selected from the group consisting of: Ar—C₀₋₆alkyl, R⁹C(O)— and R⁹SO₂;

R⁴ is R⁵C(O)—;

R⁵ is selected from the group consisting of:

methyl, especially halogenated methyl, more especially trifluoromethyl, especially C₁₋₆alkoxy and aryloxy substituted methyl, more especially phenoxy-methyl, 4-fluoro-phenoxy-methyl, especially heterocycle substituted methyl, more especially 2-thiophenyl-methyl;

ethyl, especially piperidin-1-yl-ethyl;

butyl, especially aryl substituted butyl, more especially 4-(4-methoxy)phenyl-butyl;

isopentyl;

cyclohexyl;

butenyl, especially aryl substituted butenyl, more especially 4,4-bis(4-methoxyphenyl)-but-3-enyl;

acetyl;

pentanonyl, especially 4-pentanonyl;

phenyl, especially phenyl substituted with one or more halogens, more especially 3,4-dichlorophenyl and 4-fluorophenyl, especially phenyl substituted with one or more aryloxy or C₁₋₆alkoxy groups, more especially 3,4-dimethoxy-phenyl, 3-benzyloxy-4-methoxy-phenyl, especially phenyl substituted with one or more C₁₋₆alkyl sulfonyl groups, more especially 4-methanesulfonyl-phenyl;

benzyl;

naphthalenyl, especially naphthylen-2-yl;

benzo[1,3]dioxolyl, especially benzo[1,3]dioxol-5-yl;

furanyl, especially furan-2-yl, especially nitro substituted furanyl, more especially 5-nitro-furan-2-yl, more especially halogen substituted furanyl, even more especially 5-bromo-furan-2-yl, more especially aryl substituted furanyl, even more especially 5-(4-chloro-phenyl)-furan-2-yl, 5-(4-nitrophenyl)-furan-2-yl, 5-(3-trifluoromethyl-phenyl)-furan-2-yl, more especially C₁₋₆alkyl substituted furanyl, even more especially 3-methyl-furan-2-yl, 4-methyl-furan-2-yl, 2,5-dimethyl-furan-2-yl, and 2,4-dimethyl-furan-3-yl;

tetrahydrofuranyl, especially tetrahydrofuran-2-yl;

benzofuranyl, especially benzofuran-2-yl, especially C₁₋₆alkoxy substituted benzofuranyl, more especially 7-methoxy-benzofuran-2-yl, 5-methoxy-benzofuran-2-yl, 5,6-dimethoxy-benzofuran-2-yl, 5-(2-piperazin-4-carboxylic acid tert-butyl ester-ethoxy) benzofuran-2-yl, 5-(2-morpholinoyl-ethoxy)-benzofuran-2-yl, 5-(2-piperazin-1-yl-ethoxy)benzofuran-2-yl, 5-(2-cyclohexyl-ethoxy)-benzofuran-2-yl 5-methoxy-3-methyl-benzofuran-2-yl, 4-methoxy-3-methyl-benzofuran-2-yl, and 6-methoxy-3-methyl-benzofuran-2-yl; especially halogen substituted benzofuranyl, more especially 5-fluoro-benzofuran-2-yl, 5,6-difluoro-benzofuran-2-yl, 5-fluoro-3-methyl-benzofuran-2-yl, 6-fluoro-3-methyl-benzofuran-2-yl; especially C₁₋₆alkyl substituted benzofuranyl, most especially 3-methyl-benzofuran-2-yl,3,5-dimethyl-benzofuran-2-yl, and 3-ethyl-benzofuran-2-yl;

naphtho[2,1-b]-furanyl, especially naphtho[2,1-b]-furan-2-yl, alkyl substituted naphtho(2,1-b]-furanyl, especially 1-methyl-naphtho[2,1-b]-furan-2-yl;

benzo[b]thiophenyl, especially benzo[b]thiophen-2-yl; especially C₁₋₆alkoxy substituted benzo[b]thiophenyl, more especially 5,6-dimethoxy-benzo[b]thiophen-2-yl;

quinolinyl, especially quinolin-2-yl, quinolin-3-yl, quinolin-4-yl, quinolin-6-yl, and quinolin-8-yl;

quinoxalinyl, especially quinoxalin-2-yl;

1,8 naphthyridinyl, especially 1,8 naphthyridin-2-yl;

indolyl, especially indol-2-yl, especially indol-6-yl, indol-5-yl, especially C₁₋₆alkyl substituted indolyl, more especially N-methyl-indol-2-yl;

pyridinyl, especially pyridin-2-yl, pyridin-3-yl, pyridin-5-yl, especially C₁₋₆alkyl substituted pyridinyl, more especially 2-methyl-pyridin-5-yl, and oxy-pyridinyl, especially 1-oxy-pyridin-2-yland 1-oxy-pyridin-3-yl;

furo[3,2-b]-pyridinyl, especially furo[3,2-b]-pyridin-2-yl, C₁₋₆alkyl substituted furo[3,2-b]-pyridinyl, especially 3-methyl-furo[3,2-b)-pyridin-2-yl;

thiopheneyl, especially thiophen-3-yl, also thiophen-2-yl, especially C₁₋₆alkyl substituted thiopheneyl, more especially 5-methyl-thiophen-2-yland 5-methyl-thiophen-3-yl, especially halogen substituted thiopheneyl, more especially 4,5-dibromo-thiophen-2-yl;

thieno[3,2-b]thiophene, especially thieno[3,2-b]thiophene-2-yl, more especially C₁₋₆alkyl substituted thieno[3,2-b]thiophene-2-yl, more especially 5-tert-butyl-3-methyl-thieno[3,2-b]thiophene-2-yl;

isoxazolyl, especially isoxazol-4-yl, especially C₁₋₆alkyl substituted isoxazolyl, more especially 3,5-dimethyl-isoxazol-4-yl;

oxazolyl, especially oxazol-4-yl, especially aryl substituted oxazolyl, more especially 5-methyl-2-phenyl oxazol-4-yl, especially C₁₋₆alkyl substituted oxazolyl, more especially 2-phenyl-5-trifluoromethyl-oxazol-4-yl; and

1H-benzoimidazolyl, especially 1H-benzoimidazol-5-yl.

R⁹ is selected from the group consisting of:

methyl;

ethyl, especially C₃₋₆cycloalkyl-C₀₋₆alkyl-substituted ethyl, more especially 2-cyclohexyl-ethyl;

propyl;

butyl, especially C₁₋₆butyl, more especially 3-methylbutyl;

tert-butyl, particularly when R² is R⁹OC(O);

isopentyl;

phenyl, especially halogen substituted phenyl, more especially 3,4-dichlorophenyl, 4-bromophenyl, 2-fluorophenyl, 3-fluorophenyl, 4-fluorophenyl, 2-chlorophenyl, 3-chlorophenyl, 4-chlorophenyl, especially C₁₋₆alkoxy phenyl, more especially 3-methoxyphenyl, 4-methoxyphenyl, 3,4-dimethoxyphenyl, especially cyanophenyl, more especially 2-cyanophenyl; especially C₁₋₆alkyl substituted phenyl, more especially 4-ethyl-phenyl, 2-methyl phenyl, 4-methyl phenyl, especially C₁₋₆alkyl sulfonyl substituted phenyl, more especially 4-methanesulfonyl phenyl, and 2-methanesulfonyl phenyl;

toluyl, especially Het-substituted toluyl, more especially 3-(pyridin-2-yl)toluyl;

naphthylene, especially naphthyl-2-ene;

benzoic acid, especially 2-benzoic acid;

benzo[1,3]dioxolyl, especially benzo[1,3]dioxol-5-yl;

benzo[1,2,5]oxadiazolyl, especially benzo[1,2,5]oxadiazol-4-yl;

pyridinyl, especially pyridin-2-yl, pyridin-3-yl, especially 1-oxy-pyridinyl, more especially l-oxy-pyridin-2-yl, 1-oxy-pyridin-3-yl; especially C₁₋₆alkylpyridinyl, more especially 3-methyl-pyridin-2-yl, 6-methyl-pyridin-2-yl;

thiopheneyl, especially thiophene-2-yl;

thiazolyl, especially thiazol-2-yl;

1H-imidazolyl, especially 1H-imidazol-2-yl, 1H-imidazol-4-yl, more especially C₁₋₆alkyl substituted imidazolyl, even more especially 1-methyl-1H-imidazol-2-yl, 1-methyl-1H-imidazol-4-yl, and 1,2-dimethyl-1H-imidazol-4-yl;

triazolyl, especially 1H-[1,2,4]triazolyl, more especially 1H-[1,2,4]triazol-3-yl, especially C₁₋₆alkyl substituted 1H-[1,2,4]triazolyl, more especially 5-methyl-1H-[1,2,4]triazol-3-yl; and

isoxazolyl, especially isoxazo-4-yl, especially C₁₋₆alkyl substituted isoxazolyl, more especially 3,5-dimethyl-isoxazol-4-yl.

R′ is H;

R″ is H; and

R′″ is methyl.

Most preferred are compounds of Formula I wherein:

R² is R⁹SO₂;

R⁴ is R⁵C(O);

R⁵ is selected from the group consisting of: benzofuran-2-yl, 3-methyl-benzofuran-2-yl, 5-methoxybenzofuran-2-yl, thieno[3,2-b]thiophen-2-yl, quinoxalin-2-yl, and quinolin-2-yl, preferably selected from the group consisting of: benzofuran-2-yl and thieno[3,2-b]thiophen-2-yl, most preferably benzofuran-2-yl;

R⁹ is selected from the group consisting of: pyridin-2-yl and 1-oxy-pyridin-2-yl, preferably pyridin-2-yl.

R′ is H; and

R′″ is methyl.

The following compounds of Formula I are particularly preferred embodiments of the present invention:

benzofuran-2-carboxylic acid {([(S)-3-oxo-1-(pyridine-2-sulfonyl)-azepan-4-ylcarbamoyl)-cyclohexyl}-amide;

benzofuran-2-carboxylic acid {1-[(R)-3-oxo-1-(pyridine-2-sulfonyl)-azepan-4-ylcarbamoyl)-cyclohexyl}-amide;

thieno[3,2-b]thiophene-2-carboxylic acid {-[(+/−)-3-oxo-1-(pyridine-2-sulfonyl)-azepan-4-ylcarbamoyl]-cyclohexyl}-amide;

benzofuran-2-carboxylic acid {1-[(4S,7R)-7-methyl-3-oxo-1-(pyridine-2-sulfonyl)-azepan-4-ylcarbamoyl]-cyclohexyl}-amide;

thieno[3,2-b]thiophene-2-carboxylic acid {1-[(4S,7R)-7-methyl-3-oxo-1-(pyridine-2-sulfonyl)-azepan-4-ylcarbamoyl]-cyclohexyl}-amide;

2,2,4-trideutero-benzofuran-2-carboxylic acid {1-[(S)-3-oxo-1-(pyridine-2-sulfonyl)-azepan-4-ylcarbamoyl]-cyclohexyl}-amide

2,2,4-trideutero-benzofuran-2-carboxylic acid {1-[(4S,7R)-7-methyl-3-oxo-1-(pyridine-2-sulfonyl)-azepanylcarbamoyl]-cyclohexyl}-amide;

2,2,4-trideutero-thieno[3,2-b]thiophene-2-carboxylic acid {1-[(S)-3-oxo-1-(pyridine-2-sulfonyl)-azepan-4-ylcarbamoyl]-cyclohexyl}-amide; and

2,2,4-trideutero-thieno[3,2-b]thiophene-2-carboxylic acid {1-[(4S,7R)-7-methyl-3-oxo-1-(pyridine-2-sulfonyl)-azepan-4-ylcarbamoyl]-cyclohexyl}-amide.

The following compound of Formula I is the most preferred embodiment of the present invention:

benzofuran-2-carboxylic acid {1-[(4S,7R)-7-methyl-3-oxo-1-(pyridine-2-sulfonyl)-azepan-4-ylcarbamoyl]-cyclohexyl}-amide;

Specific representative compounds of the present invention are set forth in Example 1-8.

Compared to the corresponding 5 and 6 membered ring compounds, the 7 membered ring compounds of the present invention are configurationally more stable at the carbon center alpha to the ketone.

The present invention includes deuterated analogs of the inventive compounds. A representative synthesis of deuterated analogs is shown in Scheme 8. The deuterated compounds of the present invention should exhibit superior chiral stability compared to the protonated isomer.

Where possible the present invention includes quaternary salts of the inventive compounds.

### Definitions

The present invention includes all hydrates, solvates, complexes and prodrugs of the compounds of this invention. Prodrugs are any covalently bonded compounds which release the active parent drug according to Formula I in vivo. If a chiral center or another form of an isomeric center is present in a compound of the present invention, all forms of such isomer or isomers, including enantiomers and diastereomers, are intended to be covered herein. Inventive compounds containing a chiral center may be used as a racemic mixture, an enantiomerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether existing in equilibrium or predominantly in one form.

The meaning of any substituent at any one occurrence in Formula I or any subformula thereof is independent of its meaning, or any other substituent's meaning, at any other occurrence, unless specified otherwise.

Abbreviations and symbols commonly used in the peptide and chemical arts are used herein to describe the compounds of the present invention. In general, the amino acid abbreviations follow the IUPAC-IUB Joint Commission on Biochemical Nomenclature as described in Eur. J. Biochem., 158, 9 (1984).

“Proteases” are enzymes that catalyze the cleavage of amide bonds of peptides and proteins by nucleophilic substitution at the amide bond, ultimately resulting in hydrolysis. Such proteases include: cysteine proteases, serine proteases, aspartic proteases, and metaloproteases. The compounds of the present invention are capable of binding more strongly to the enzyme than the substrate and in general are not subject to cleavage after enzyme catalyzed attack by the nucleophile. They therefore competitively prevent proteases from recognizing and hydrolyzing natural substrates and thereby act as inhibitors.

The term “amino acid” as used herein refers to the D- or L-isomers of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine.

A representation of an element is understood to include all isotopes of that element. Thus, for example, the term “H” includes all isotopes of hydrogen, including deuterium.

“C₁₋₆alkyl” as applied herein is meant to include substituted and unsubstituted methyl, ethyl, n-propyl, isopropyl, n-butyl, isobutyl and t-butyl, pentyl, n-pentyl, isopentyl, neopentyl and hexyl and the simple aliphatic isomers thereof. C₁₋₆alkyl may be optionally substituted by a moiety selected from the group consisting of: OR¹⁴, C(O)R¹⁴, SR¹⁴, S(O)R¹⁴, NR¹⁴₂, R¹⁴NC(O)OR⁵, CO₂R¹⁴, CO₂NR¹⁴₂, N(C═NH)NH₂, Het, C₃₋₆cycloalkyl, and Ar; where R⁵ is selected from the group consisting of: H, C₁₋₆alkyl, C₂₋₆alkenyl, C₂₋₆alkyl, C₃₋₆cycloalkyl-C₀₋₆alkyl, Ar—C₀₋₆alkyl and Het-C₀₋₆alkyl; and R¹⁴ is selected from the group consisting of: H, C₁₋₆alkyl, Ar—C₀₋₆alkyl, and Het-C₀₋₆alkyl;

“C₃₋₆cycloalkyl” as applied herein is meant to include substituted and nsubstituted cyclopropane, cyclobutane, cyclopentane and cyclohexane.

“C₂₋₆ alkenyl” as applied herein means an alkyl group of 2 to 6 carbons wherein a carbon-carbon single bond is replaced by a carbon-carbon double bond. C₂₋₆alkenyl includes ethylene, 1-propene, 2-propene, 1-butene, 2-butene, isobutene and the several isomeric pentenes and hexenes. Both cis and trans isomers are included.

“C₂₋₆alkynyl” means an alkyl group of 2 to 6 carbons wherein one carbon-carbon single bond is replaced by a carbon-carbon triple bond. C₂₋₆ alkynyl includes acetylene, 1-propyne, 2-propyne, 1-butyne, 2-butyne, 3-butyne and the simple isomers of pentyne and hexyne.

“C₂₋₆alkanonyl” means an acyl group of 2 to 6 carbons wherein one non-terminal carbon is substituted by the ═O group. C₂₋₆alkanonyl includes, for example, acetyl, 1- and 2-propanonyl, 1-, 2-, and 3-butanonyl, 1-, 2-, 3- and 4-pentanonyl and 1-, 2-, 3-, 4- and 5-hexanonyl.

“Halogen” means F, Cl, Br, and I.

“tAr” or “aryl” means phenyl or naphthyl, optionally substituted by one or more of Ph-C₀₋₆alkyl; Het-C₀₋₆alkyl; C₁₋₆alkoxy; Ph-C₀₋₆alkoxy; Het-C₀₋₆alkoxy; OH, (CH₂)₁₋₆NR¹⁵R¹⁶; O(CH₂)₁₋₆NR¹⁵R¹⁶; C₁₋₆alkyl, OR¹⁷, N(R¹⁷)₂, SR¹⁷, CF₃, NO₂, CN, CO₂R¹⁷, CON(R¹⁷)₂, F, Cl, Br or I; where R¹⁵ and R¹⁶ are H, C₁₋₆alkyl, Ph—C₀₋₆alkyl, naphthyl-C₀₋₆alkyl or Het-C₀₋₆alkyl; and R¹⁷ is phenyl, naphthyl, or C₁₋₆alkyl.

As used herein “Het” or “heterocyclic” represents a stable 5- to 7-membered monocyclic, a stable 7- to 10-membered bicyclic, or a stable 11- to 18-membered tricyclic heterocyclic ring which is either saturated or unsaturated, and which consists of carbon atoms and from one to three heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure, and may optionally be substituted with one or two moieties selected from C₀₋₆Ar, C₁₋₆alkyl, OR¹⁷, N(R¹⁷)₂, SR¹⁷, CF₃, NO₂, CN, CO₂R¹⁷, CON(R¹⁷), F, Cl, Br and I, where R¹⁷ is phenyl, naphthyl, or C₁₋₆alkyl. Examples of such heterocycles include piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, pyridinyl, 1-oxo-pyridinyl, pyrazinyl, oxazolidinyl, oxazolinyl, oxazolyl, isoxazolyl, morpholinyl, thiazolidinyl, thiazolinyl, thiazolyl, quinuclidinyl, indolyl, quinolinyl, quinoxalinyl, isoquinolinyl, benzimidazolyl, benzopyranyl, benzoxazolyl, furanyl, benzofuranyl, thiophenyl, benzo[b]thiophenyl, thieno[3,2-b]thiophenyl, benzo[1,3]dioxolyl, 1,8 naphthyridinyl, pyranyl, tetrahydrofuranyl, tetrahydropyranyl, thienyl, benzoxazolyl, thiamorpholinyl sulfoxide, thiamorpholinyl sulfone, and oxadiazolyl, as well as triazolyl, thiadiazolyl, oxadiazolyl, isothiazolyl, imidazolyl, pyridazinyl, pyrimidinyl, triazinyl and tetrazinyl which are available by routine chemical synthesis and are stable. The term heteroatom as applied herein refers to oxygen, nitrogen and sulfur.

Here and throughout this application the term C₀ denotes the absence of the substituent group immediately following; for instance, in the moiety ArC₀₋₆alkyl, when C is 0, the substituent is Ar, e.g., phenyl. Conversely, when the moiety ArC₀₋₆alkyl is identified as a specific aromatic group, e.g., phenyl, it is understood that the value of C is 0.

Certain radical groups are abbreviated herein. t-Bu refers to the tertiary butyl radical, Boc refers to the t-butyloxycarbonyl radical, Fmoc refers to the fluorenylmethoxycarbonyl radical, Ph refers to the phenyl radical, Cbz refers to the benzyloxycarbonyl radical.

Certain reagents are abbreviated herein. m-CPBA refers to 3-chloroperoxybenzoic acid, EDC refers to N-ethyl-N′(dimethylaminopropyl)-carbodiimide, DMF refers to dimethyl formamide, DMSO refers to dimethyl sulfoxide, HBTU refers to O-Benzotriazol-1-yl-N,N,N′,N′-tetramethyluronium hexafluorophosphate, TEA refers to triethylamine, NMM refers to N-methylmorpholine, TFA refers to trifluoroacetic acid, and TBF refers to tetrahydrofuran.

### Methods of Preparation

Compounds of the general formula I may be prepared in a fashion analogous to that outlined in Schemes 1-11.

Alkylation of tert-butyl N-alkylcarbamate (1) with a base such as sodium hydride and 5-bromo-1-pentene provides the diene 2. Treatment of 2 with either 2,6-diisopropylphenylimido neophylidene molybenum bis(tert-butoxide) or bis(tricyclohexylphosphine)benzylidine ruthenium (IV) dichloride olefin metathesis catalysts developed by Grubbs provides the azepine 3. Epoxidation of 3 with standard oxidizing agents common to the art such as m-CPBA provide the epoxide 4. Nucleophilic epoxide ring opening may be effected with a reagent such as sodium azide to provide the azido alcohol (not shown) which may be reduced to the amino alcohol 5 under conditions common to the art such as 1,3-propanedithiol and triethylamine in methanol or with hydrogen gas in the presence of a catalyst such as palladium on carbon. Acylation of 5 with an acid such as Cbz-amino-cyclohexanecarboxylic acid in the presence of a coupling agent such as EDC followed by removal of the BOC protecting group under acidic conditions provides the amine salt 6. Coupling of 6 with Cbz-amino-cyclohexanecarboxylic acid may be effected with a coupling agent such as EDC to provide the intermediate alcohol (not shown) which was oxidized with an oxidant such as pyridine sulfur trioxide complex in DMSO and triethylamine to provide the ketone 7.

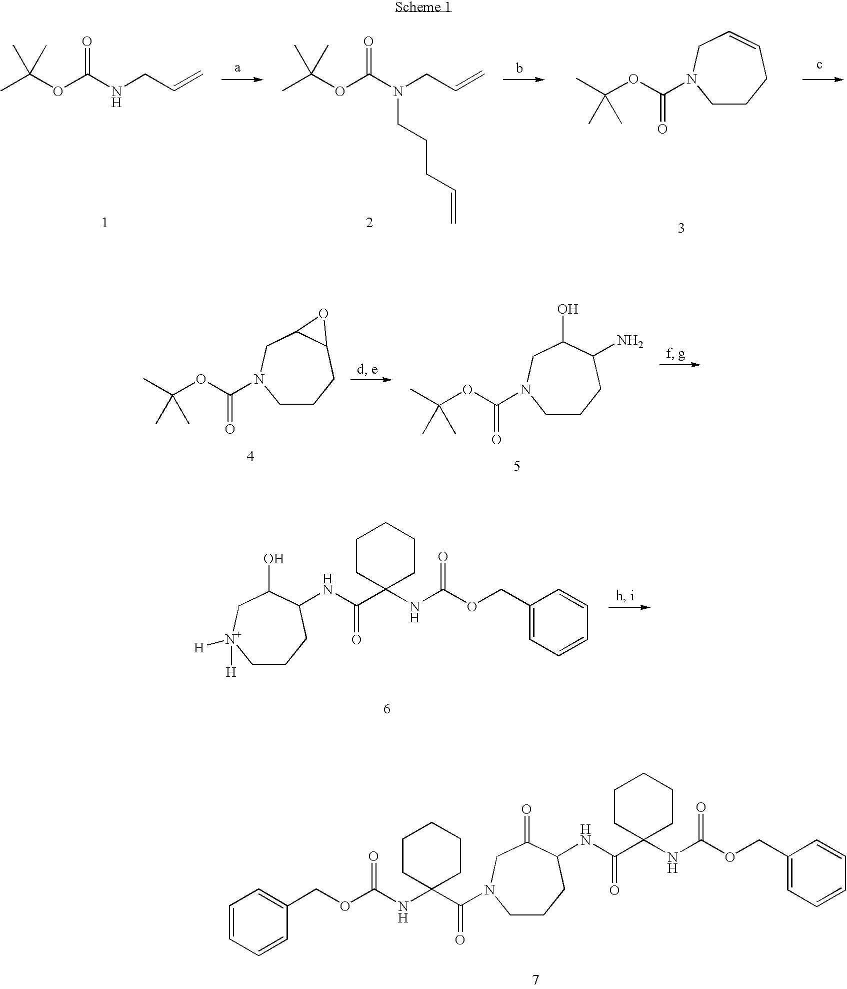

Reagents and conditions: a.) NaH, 5-bromo-1-pentene, DMF; b.) 2,6-diisopropylphenylimide neophylidene molybenum bis(tert-butoxide) or bis(tricyclohexylphosphine)benzylidine ruthenium (IV) dichloride catalyst, toluene c.) m-CPBA, CH₂Cl₂; d.) NaN₃, CH₃OH, H₂O, NH₄Cl; e.) 10% Pd/C, H₂, f.) Cbz-amino-cyclohexanecarboxylic acid, EDC, CH₂C₂; g.) HCl, EtOAc; h.) Cbz-amino-cyclohexanecarboxylic acid, EDC, CH₂Cl₂; i.) pyridine sulfur trioxide complex, DMSO, TEA.

Compounds of the general formula I wherein R¹ and R² are amides may be prepared in the general fashion outlined in Scheme 2. Alkylation of N-Cbz alkyl amine (8) with a base such as sodium hydride and 5-bromo-1-pentene provides the diene 9. Treatment of 9 with bis(tricyclohexylphosphine)benzylidine ruthenium(IV)dichloride olefin metathesis catalyst developed by Grubbs provides the azepine 10. Epoxidation of 10 with standard oxidizing agents common to the art such as M-CPBA provide the epoxide 11. Nucleophilic epoxide ring opening may be effected with a reagent such as sodium azide to provide the azido alcohol (not shown) which may be reduced to the amino alcohol 12 with a reducing agent such as propanedithiol in the presence of triethylamine. Acylation of 12 with N-Boc-amino-cyclohexanecarboxylic acid and a coupling agent such as EDC followed by removal of the Cbz protecting group under hydrogenolysis conditions provides the amine 13. Coupling of 13 with a carboxylic acid was effected with a coupling agent such as EDC followed by removal of the acid labile N-Boc protecting group with an acid such as HCl or TFA provides intermediate 14. Acylation of 14 may be effected with a carboxylic acid in the presence of a coupling agent common to the art such as EDC to give the intermediate alcohol (not shown) which is oxidized with an oxidant such as pyridine sulfur trioxide complex in DMSO and triethylamine to provide the ketone 15.

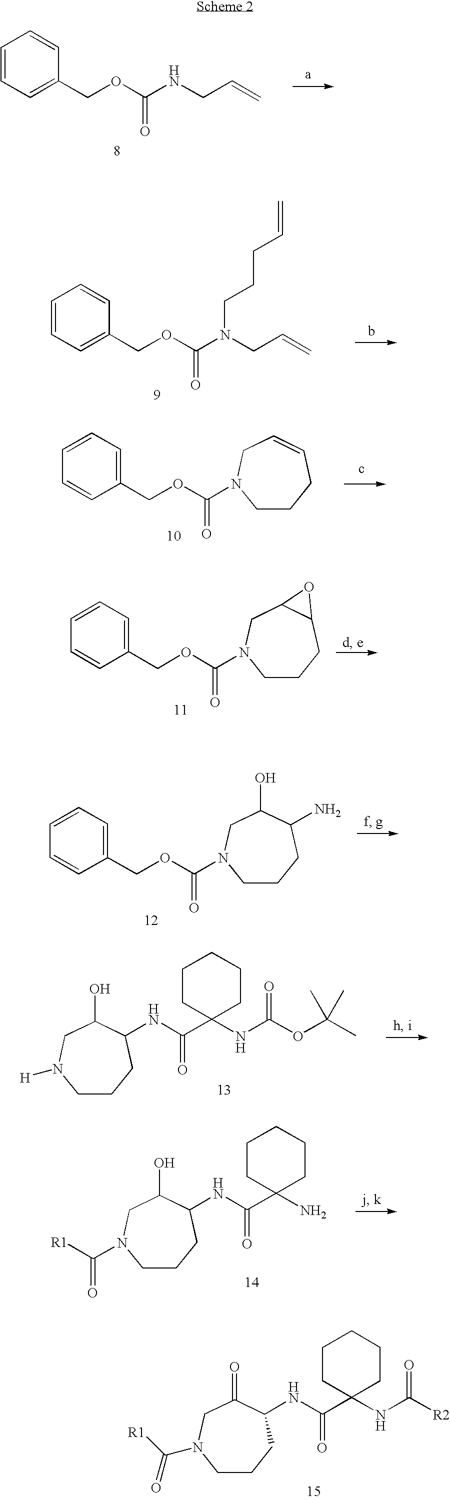

Reagents and conditions: a.) NaH, 5-bromo-1-pentene, DMF; b.) bis(tricyclohexylphosphine)benzylidine ruthenium (IV) dichloride catalyst, CH₂Cl₂; c.) m-CPBA, CH₂Cl₂; d.) NaN₃, CH₃OH, H₂O, NH₄Cl; e.) propanedithiol, CH₃OH, TEA; f.) Boc-amino-cyclohexanecarboxylic acid, EDC, CH₂Cl₂; g.) 10% Pd/C, H₂; h.) R₁CO₂H, EDC, CH₂Cl₂ or R₁COCl, CH₂Cl₂; i.) HCl/EtOAc; J.) R₂CO₂H, EDC, CH₂Cl₂; k.) pyridine sulfur trioxide complex, DMSO, TEA.

Compounds of the general formula I wherein R² is an alkyl, urea or sulphonamide group and R¹ is an amide may be prepared in the general fashion outlined in Scheme 3. Reductive amination of 13 may be effected by treatment with an aldehyde followed by a reducing agent such as sodium triacetoxyborohydride. Subsequent deprotection of the N-Boc group under acidic conditions provides the amine salt 16. Coupling of 16 with an acid chloride or with a carboxylic acid in the presence of a coupling agent common to the art such as EDC followed by oxidation of the intermediate alcohol (not shown) with an oxidant such as pyridine sulfur trioxide complex provides the ketone 17. Alternatively, treatment of amine 13 with an isocyanate followed by deprotection of the N-Boc group provides the amine salt 18. Acylation and oxidation provides the ketone 19. Further derivatization of amine 13 may be effected by treatment with a sulphonyl chloride followed by deprotection of the N-Boc group to provide the amine salt 20. Acylation and oxidation provides the ketone 21.

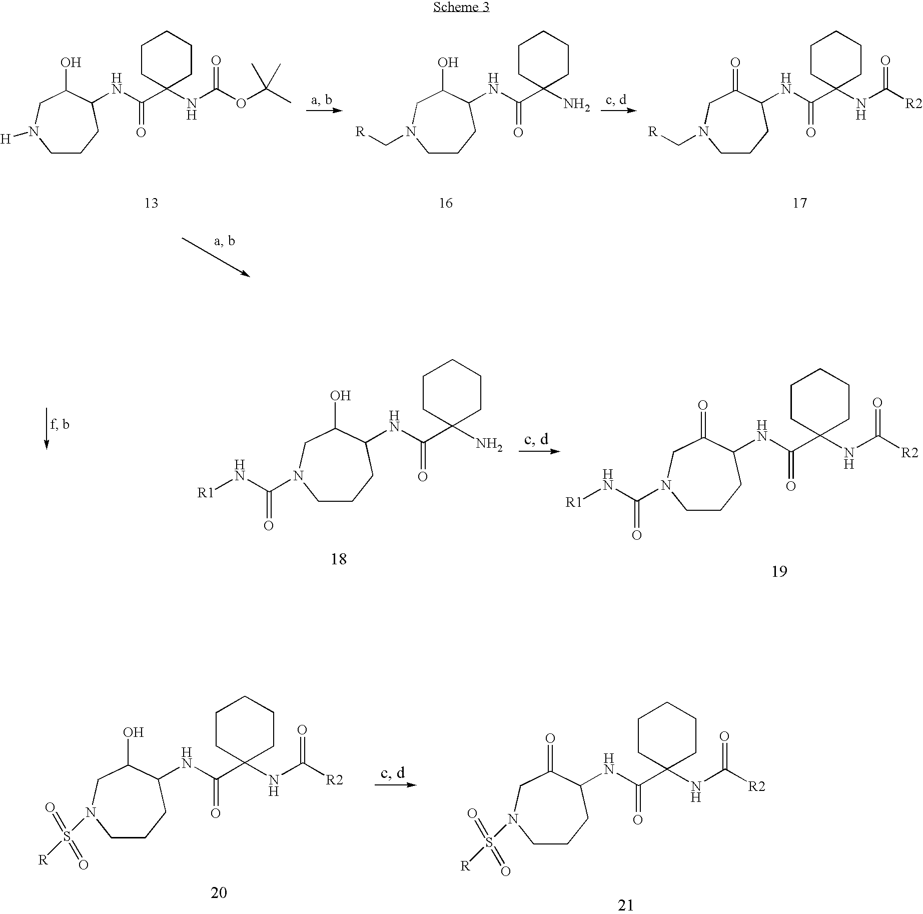

Reagents and conditions: a.) R₁CHO, NaBH(OAc)₃; b.) HCl; c.) R₂CO₂H, EDC, CH₂Cl₂; d.) pyridine sulfur trioxide complex, DMSO, TEA; e.) R₁NCO, base; f.) R₁SO₂Cl, TEA, CH₂Cl₂.

The individual diastereomers of benzofuran-2-carboxylic acid {(S)-3-methyl-1-[(2,2′,4-trideuterio)-3-oxo-1-(pyridine-2-sulfonyl)-azepanylcarbamoyl]-butyl}amide 31 and 32 may be prepared as outlined in Scheme 4. Alkylation of alkyl-carbamic acid benzyl ester 22 with 5-bromo-1-pentene in the presence of a base such as sodium hydride provides the diene 23. Treatment of diene 23 with bis(tricyclohexylphosphine)benzylidine ruthenium (IV) dichloride developed by Grubbs provides the 2,3,4,7-tetrahydro-azepine-1-carboxylic acid benzyl ester 24. Epoxidation of azepine 24 may be effected with standard oxidizing agents common to the art such as M-CPBA to provide epoxide 25. Nucleophilic epoxide ring opening of 25 may be effected with a reagent such as sodium azide to provide the azido alcohol (not shown). The intermediate azido alcohol may be reduced to the amino alcohol 26 under conditions common to the art such as 1,3-propanedithiol and triethylamine in methanol or with triphenylphosphine in tetrahydrofuran and water. Acylation of 26 may be effected with an acid such as N-Boc-amino-cyclohexanecarboxylic acid in the presence of a coupling agent such as EDC. Removal of the benzyloxycarbonyl protecting group with hydrogen gas in the presence of 10% Pd/C provides the amine 27. Treatment of the amine 27 with 2-pyridinesulphonyl chloride in the presence of triethylamine or saturated sodium bicarbonate and CH₂Cl₂ followed by removal of the tert-butoxycarbonyl protecting group under acidic conditions provides 28. Coupling of 28 with benzofuran-2-carboxylic acid may be effected with a coupling agent such as EDC to provide intermediate alcohol 29. Alcohol 29 may be oxidized with an oxidant such as sulfur trioxide pyridine complex in DMSO and triethylamine to provide the ketone 30 as a mixture of diastereomers.

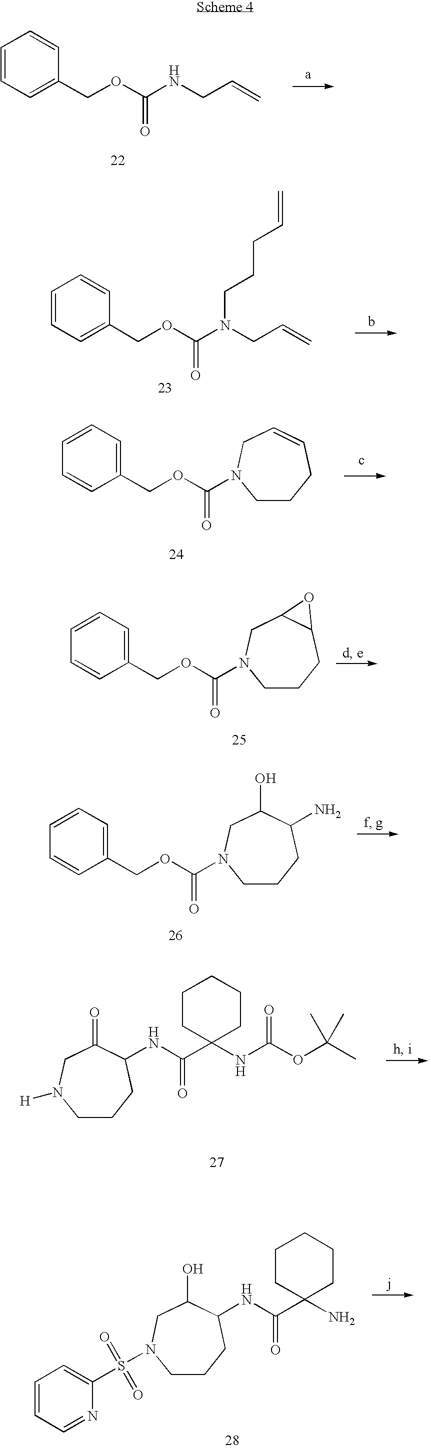

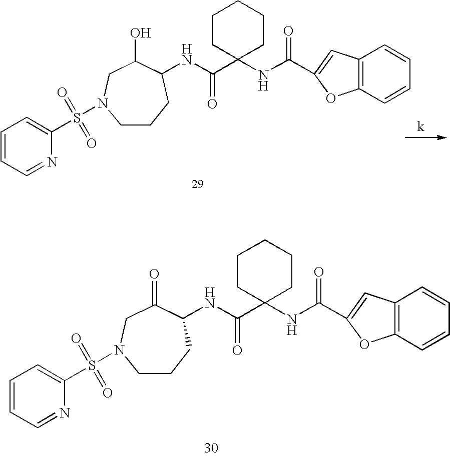

Reagents and Conditions: a.) NaH, 5-bromo-1-pentene, DMF; b.) bis(tricyclohexylphosphine)benzylidine ruthenium (IV) dichloride, CH₂Cl₂; c.) m-CPBA, CH₂Cl₂; d.) NaN₃, CH₂OH, H₂O, NH₄Cl; e.) 1,3-propanedithiol, TEA, methanol; f.) N-Boc-amino-cyclohexanecarboxylic acid, EDC, CH₂Cl₂; g.) 10% Pd/C, H; h.) 2-pyridinesulphonyl chloride, TEA, CH₂Cl₂; i.) 4 N HCl/dioxane, methanol; j.) benzofuran-2-carboxylic acid, EDC, CH₂Cl₂; k.) pyridine sulfur trioxide complex, DMSO, TEA

The quaternized, 4-amino-azepan-3-one compounds of the present invention may be conveniently prepared according to Scheme 5. The skilled artisan will understand from Scheme 5 how to make any of the quaternized, 4-amino-azepan-3-one compounds of the present invention. Reductive amination of 13 may be effected by treatment with an aldehyde followed by a reducing agent such as sodium triacetoxyborohydride. Subsequent deprotection of the N-Boc group under acidic conditions—provides the amine salt 16. Treatment of 16 with an acid chloride or with a carboxylic acid in the presence of a coupling agent common to the art such as EDC followed by oxidation of the intermediate alcohol (not shown) with an oxidant such as pyridine sulfur trioxide complex provides the ketone 17. Quaternization of the amine of 17 may be effected by treatment with an alkylating agent such as iodomethane to provide the quaternary amine salt 41.

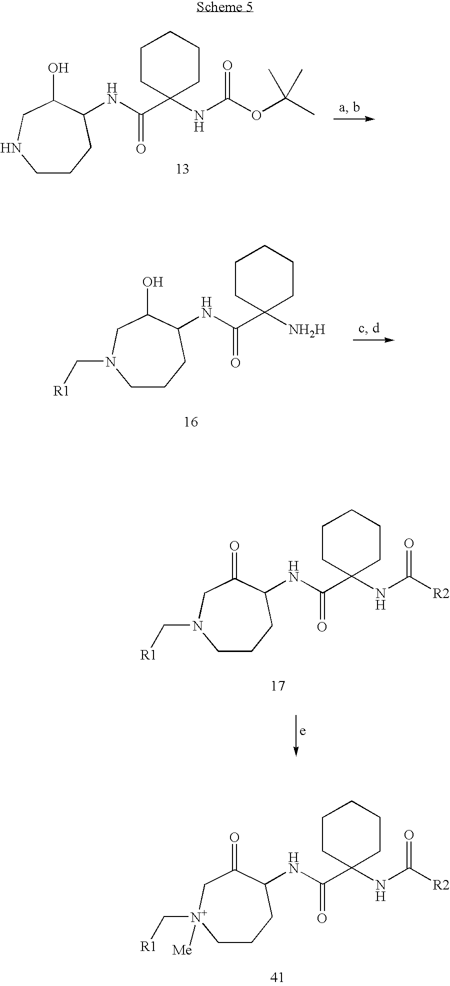

Reagents and conditions: a.) R₁CHO, NaBH(OAc)₃; b.) HCl; c.) R₂CO₂H, EDC, CH₂Cl₂; d.) pyridine sulfur trioxide complex, DMSO, TEA; e.) iodomethane

The 6-methyl-4-amino-azepan-3-one compounds of the present invention may be conveniently prepared according to Scheme 6,2-Methyl-pent-4-enoic acid ethyl ester is converted to a N-2-pyridinesulfonyl-azapine by reduction to the aldehyde, reductive amination with alkylamine, sulfonylation with 2-pyridyl sulfonyl chloride, and olefin metathesis with Grubbs' catalyst. Epoxidation with mCPBA affords a mixture of epoxides that are separable by column chromatography. The syn epoxide is converted into an amino alcohol by opening with sodium azide followed by reduction with triphenylphosphine. Acylation of the free amine with Boc-amino-cyclohexanecarboxylic acid and a coupling reagent such as HBTU or EDC, followed by deprotection of the Boc group with HCl, and acylation with a variety of aromatic carboxylic acids and coupling reagents such as HBTU or EDC gives the intermediate alcohols. Final oxidation with Dess-Martin periodinane and HPLC affords the desired ketones.

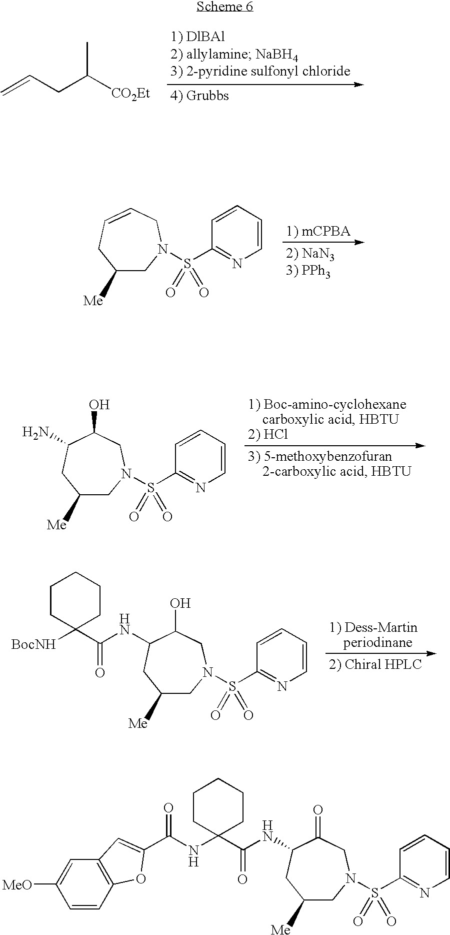

The 7-methyl-4-amino-azepan-3-one compounds of the present invention may be conveniently prepared according to Scheme 7. Carbobenyzloxy-D-alaninol (Cbz-D-alaminol) is first converted to an iodide, then is reacted with alkyl Grignard with a copper (I) catalyst or a similar alkyl organometallic reagent. The amine is then alkylated with alkyl iodide. Grubbs' catalyst is then used to form the azapine ring by ring closing metathesis. Epoxidation of the alkene followed by separation of the diastereomers followed by opening of the epoxide of the minor component with sodium azide provides the intermediate azido alcohol. Reduction of the azide followed by acylation of the amine with a 1N-protected cycloalkane-carboxylic acid such as N-Boc-1-amino-cyclohexane-1-carboxylic acid and a peptide coupling reagent such as HBTU or EDC, followed by deprotection of the Cbz gives the intermediate secondary amine, which is then sulfonylated with an sulfonyl chloride. Deprotection of the Boc group followed by acylation with a carboxylic acid and a peptide coupling reagent such as HBTU or EDC, and final oxidation of the secondary alcohol to the ketone with an oxidant such as Dess-Martin periodinane or sulfurtrioxide pyridine complex with triethylamine provides the desired product.

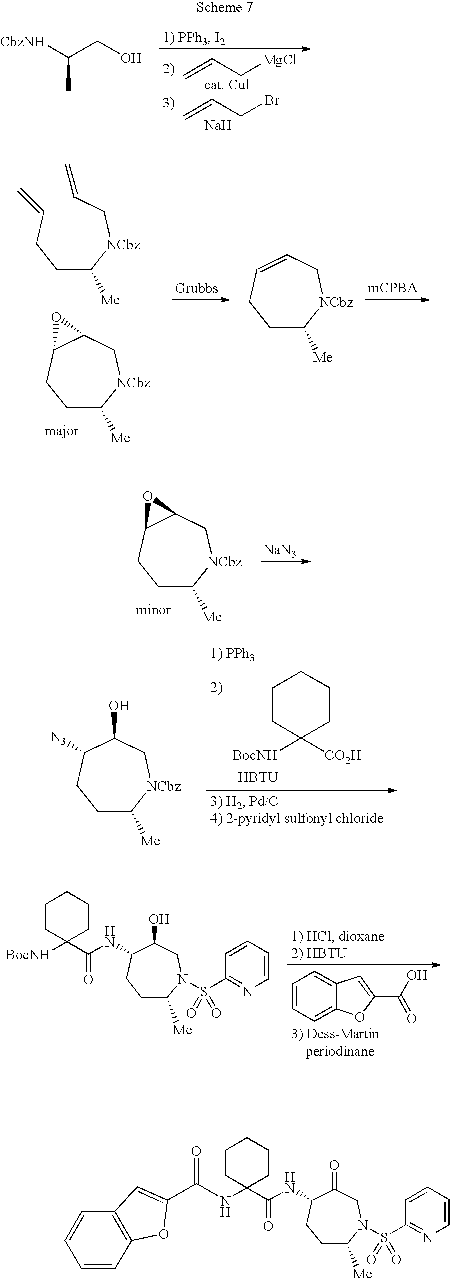

The 2,2-4-trideutero-4-amino-azepan-3-one compounds of the present invention may be conveniently prepared according to Scheme 8. Deuterated inhibitors can be prepared from the parent inhibitors such as benzofuran-2-carboxylic acid {(S)-3-methyl-1-[(4S,7R)-7-methyl-3-oxo-1-(pyridine-2-sulfonyl)-azepan-4-ylcarbamoyl]-butyl}-amide by treating with a base such as triethyl amine and stirring for several days in a deteurated protic solvent such as CD₃OD: D₂O.

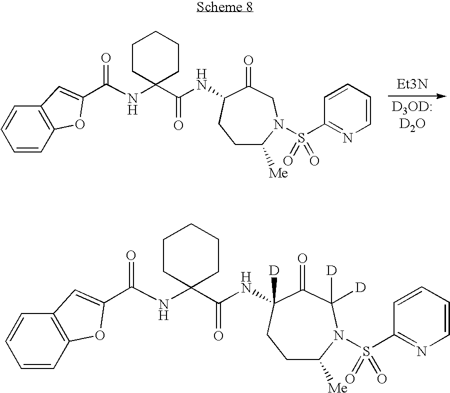

The 7-methyl-4-amino-azepan-3-one compounds of the present invention may be conveniently prepared according to Scheme 9. Intermediate (S)-3-Cyclohexyl-N-((3S,4S,7R)-3-hydroxy-7-methyl-azepan-4-yl)-2-methyl-propionamide, as described in Scheme 3, is reductively aminated with an aldehyde or a ketone such as propionaldehyde, then treated with a reducing agent such as sodium borohydride. Deprotection of the Boc group followed by acylation with an acylating agent such as 2-furan carboxylic acid, UBTU, NMM, and final oxidation of the secondary alcohol to the ketone with an oxidant such as sulfur trioxide-pyridine provides the desired products.

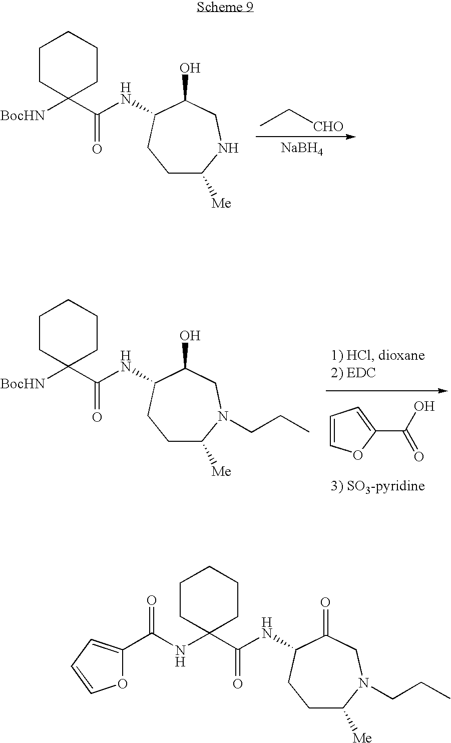

The 7-methyl-4-amino-azepan-3-one compounds of the present invention may be conveniently prepared according to Scheme 10. Intermediate 1-Methyl-cyclohexanecarboxylic acid ((3S,4S,7R)-3-hydroxy-7-methyl-azepan-4-yl)-amide, as described in Scheme 3, is acylated with an isocyanate such as (S)-(−)-2-isocyanato-4-methylvaleric acid methyl ester. Deprotection of the Boc group followed by acylation with an acylating agent such as benzofaran-2-carboxylic acid, BBTU, NMM, and final oxidation of the secondary alcohol to the ketone with an oxidant such as Dess-Martin periodinane or sulfur trioxide-pyridine provides the desired products.

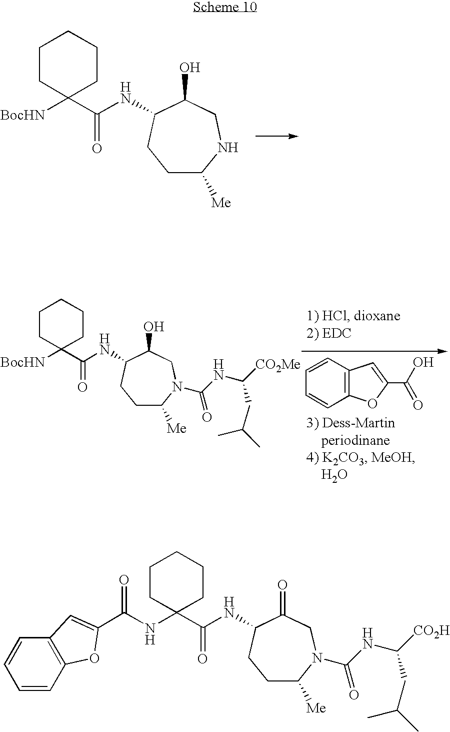

The 5-methyl-4-amino-azepan-3-one compounds of the present invention may be conveniently prepared according to Scheme 11. The synthesis of the C-5 methyl azepinone, 4,5 (R,S)-benzofuran-2-carboxylic acid {(S)-3-methyl-1-[5-methyl-3-oxo-1-(pyridine-2-sulfonyl)-azepan-4-ylcarbamoyl]-butyl}amide, (Example 61) is outlined below in Scheme 7. Michael addition of nitromethane to ethyl crotonate 7-1 followed by reduction of the intermediate ester with a reducing agent such as diisobutyl aluminum hydride (Dibal-H) provides the aldehyde 7-2. Reductive amination of 7-2 with N-benzyl ethanolamine in the presence of a reducing agent such as sodium triacetoxyborohydride provides the nitro-alcohol 7-3. Oxidation of 7-3 using an oxidant common to the art such as DMSO and oxalyl chloride followed by treatment of the crude intermediate aldehyde with a base such as triethylamine effects the nitro-aldol reaction to give the azepanol 7-4. Reduction of the nitro group with zinc in the presence of hydrochloric acid followed by coupling of the resulting amine with N-Boc-amino-cyclohexanecarboxylic acid in the presence of a coupling agent common to the art such as EDC provides intermediate 7-5. Reductive removal of the N-benzyl moiety with hydrogen gas in the presence of a catalyst such as 10% Pd on carbon followed by sulfonylation with a sulfonyl chloride in the presence of a base such as N-methylmorpholine or triethyl amine provides the sulfonamide intermediate 7-6. Removal of the N-Boc protecting group under acidic conditions followed by coupling of the resulting amine salt with benzofuran-2-carboxylic acid and oxidation of the alcohol with an oxidant common to the art such as pyridine sulfur trioxide complex or Dess-Martin periodinane provides the ketone 7. The individual diastereomers of 7-7 may be separated by HPLC methods to provide diastereomers 7-8,7-9, 7-10 and 7-11.

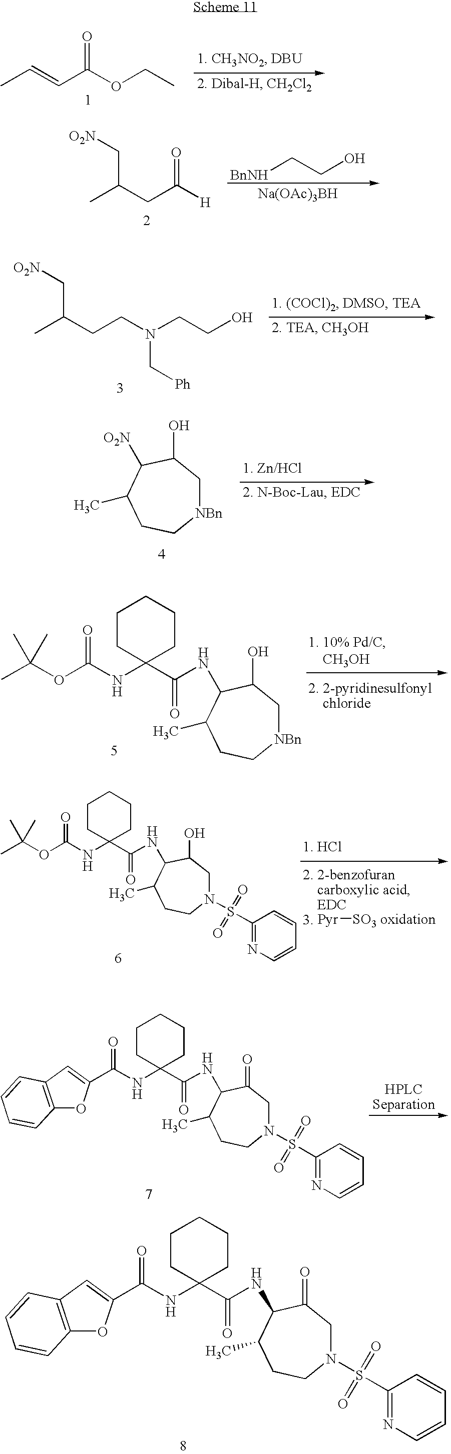

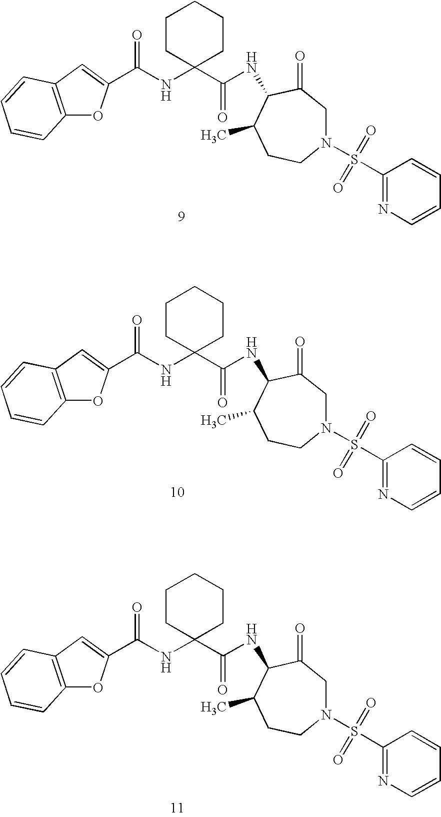

The starting materials used herein are commercially available amino acids or are prepared by routine methods well known to those of ordinary skill in the art and can be found in standard reference books, such as the COMPENDIUM OF ORGANIC SYNTHETIC METHODS, Vol. I-VI (published by Wiley-Interscience).

Coupling methods to form amide bonds herein are generally well known to the art. The methods of peptide synthesis generally set forth by Bodansky et al., THE PRATICE OF PEPTIDE SYNO SIS, Springer-Verlag, Berlin, 1984; E. Gross and J. Meienhofer, THE PEPTIDES, Vol. 1, 1-284 (1979); and J. M. Stewart and J. D. Young, SOLID PHASE PEPTIDE SYNTHESIS, 2d Ed., Pierce Chemical Co., Rockford, Ill., 1984 are generally illustrative of the technique and are incorporated herein by reference.

Synthetic methods to prepare the compounds of this invention frequently employ protective groups to mask a reactive functionality or minimize unwanted side reactions. Such protective groups are described generally in Green, T. W, PROTECTIVE GROUPS IN ORGANIC SYNTHESIS, John Wiley & Sons, New York (1981). The term “amino protecting groups” generally refers to the Boc, acetyl, benzoyl, Fmoc and Cbz groups and derivatives thereof as known to the art. Methods for protection and deprotection, and replacement of an amino protecting group with another moiety are well known.

Acid addition salts of the compounds of Formula I are prepared in a standard manner in a suitable solvent from the parent compound and an excess of an acid, such as hydrochloric, hydrobromic, hydrofluoric, sulfuric, phosphoric, acetic, trifluoroacetic, maleic, succinic or methanesulfonic. Certain of the compounds form inner salts or zwitterions which may be acceptable. Cationic salts are prepared by treating the parent compound with an excess of an alkaline reagent, such as a hydroxide, carbonate or alkoxide, containing the appropriate cation; or with an appropriate organic amine. Cations such as Li⁺, Na⁺, K⁺, Ca⁺⁺, Mg⁺⁺ and NH₄⁺ are specific examples of cations present in pharmaceutically acceptable salts. Halides, sulfate, phosphate, alanoates (such as acetate and trriluoroacetate), benzoates, and sulfonates (such as mesylate) are examples of anions present in pharmaceutically acceptable salts. Quaternary ammonium salts are prepared by treating a parent amine compound with an excess of alkyl halide, such as methyl iodide.

This invention also provides a pharmaceutical composition which comprises a compound according to Formula I and a pharmaceutically acceptable carrier, diluent or excipient. Accordingly, the compounds of Formula I may be used in the manufacture of a medicament. Pharmaceutical compositions of the compounds of Formula I prepared as hereinbefore described may be formulated as solutions or lyophilized powders for parenteral administration. Powders may be reconstituted by addition of a suitable diluent or other pharmaceutically acceptable carrier prior to use. The liquid formulation may be a buffered, isotonic, aqueous solution. Examples of suitable diluents are normal isotonic saline solution, standard 5% dextrose in water or buffered sodium or ammonium acetate solution. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride or sodium citrate.

Alternately, these compounds may be encapsulated, tableted or prepared in an emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

For rectal administration, the compounds of this invention may also be combined with excipients such as cocoa butter, glycerin, gelatin or polyethylene glycols and molded into a suppository.

### Novel Intermediates

Referring to the methods of preparing the compounds of Formula I set forth in Schemes 1-11 above, the skilled artisan will appreciate that the present invention includes all novel intermediates required to make the compounds of Formula I. In particular, the present invention provides the compounds of Formula II:

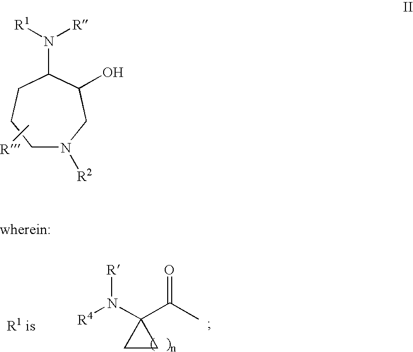

R² is selected from the group consisting of: H, C₁₋₆alkyl, C₃₋₆cycloalkyl-C₀₋₆alkyl, Ar—C₀₋₆alkyl, Het-C₀₋₆alkyl, R⁹C(O)—, R⁹C(S)—, R⁹SO₂—, R⁹OC(O)—, R⁹R¹¹NC(O)—, R⁹R¹¹NC(S)—, R⁹(R¹¹)NSO₂—

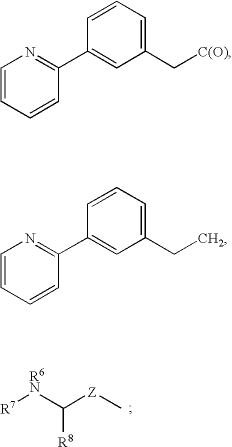

and R⁹SO₂R¹¹NC(O)—;

R⁴ is selected from the group consisting of: H, C₁₋₆alkyl, C₃₋₆cycloalkyl-C₀₋₆alkyl, Ar—C₀₋₆alkyl, Het-C₀₋₆alkyl, R⁵C(O)—, R⁵C(S)—, R⁵SO₂—, R⁵OC(O)—, R⁵R¹²NC(O)—, and R⁵R¹²NC(S)—;

R⁵ is selected from the group consisting of: H, C₁₋₆alkyl, C₂₋₆alkenyl, C₂₋₆alkynyl, C₃₋₆cycloalkyl-C₀₋₆alkyl, C₂₋₆alkanonyl, Ar—C₀₋₆alkyl and Het-C₀₋₆alkyl;

R⁶ is selected from the group consisting of: H, C₁₋₆alkyl, Ar—C₀₋₆alkyl, or Het-C₀₋₆alkyl;

R⁷ is selected from the group consisting of: H, C₁₋₆alkyl, C₃₋₆cycloalkyl-C₀₋₆alkyl, Ar—C₀₋₆alkyl, Het-C₀₋₆alkyl, R¹⁰C(O)—, R¹⁰C(S)—, R¹⁰SO₂—, R¹⁰OC(O)—, R¹⁰R¹³NC(O)—, and R¹⁰R¹³NC(S)—;

R⁸ is selected from the group consisting of: H, C₁₋₁₆alkyl, C₂₋₆alkenyl, C₂₋₆alkynyl, HetC₀₋₆alkyl and ArC₀₋₆alkyl;

R⁹ is selected from the group consisting of: C₁₋₆alkyl, C₃₋₆cycloalkyl-C₀₋₆alkyl, Ar—C₀₋₆alkyl, Ar—COOH, and Het-C₀₋₆alkyl;

R¹⁰ is independently selected from the group consisting of: C₁₋₆alkyl, C₃₋₆cycloalkyl-C₀₋₆alkyl, Ar—C₀₋₆alkyl and Het-C₀₋₆alkyl;

R¹¹ is selected from the group consisting of: H, C₁₋₆alkyl, Ar—C₀₋₆alkyl, and Het-C₀₋₆alkyl;

R¹² is selected from the group consisting of: H, C₁₋₆alkyl, Ar—C₀₋₆alkyl, and Het-C₀₋₆alkyl;

R¹³ is selected from the group consisting of: H, C₁₋₆alkyl, Ar—C₀₋₆alkyl, and Het-C₀₋₆alkyl;

R′ is selected from the group consisting of: H, C₁₋₆alkyl, Ar—C₀₋₆alkyl, and Het-C₀₋₆alkyl;

R″ is selected from the group consisting of: H, Cl ₆alkyl, Ar—C₀₋₆alkyl, or Het-C₀₋₆alkyl;

R′″ is selected from the group consisting of: H, C₁₋₆alkyl, C₃₋₆cycloalkyl-C₀₋₆alkyl, Ar—CO₆alkyl, and Het-C₀₋₆alkyl;

Z is selected from the group consisting of: C(O) and CH₂;

n is an integer of from 1 to 5;

and pharmaceutically acceptable salts, hydrates and solvates thereof.

The following compounds are preferred novel intermediates:

benzofuran-2-carboxylic acid {1-[(+/−)-3-hydroxy-1-(pyridine-2-sulfonyl)-azepan-4-ylcarbamoyl]-cyclohexyl}-amide;

thieno[3,2-b]thiophene-2-carboxylic acid {1-[(+/−)-3-hydroxy-1-(pyridine-2-sulfonyl)-azepan-4-ylcarbamoyl]-cyclohexyl}-amide;

benzofuran-2-carboxylic acid {1-[(3S,4S,7R)-3-hydroxy-7-methyl-1-(pyridine-2-sulfonyl)-azepan-4-ylcarbamoyl]-cyclohexyl}-amide; and

thieno[3,2-b]thiophene-2-carboxylic acid {1-[(3S,4S,7R)-3-hydroxy-7-methyl-1-(pyridine-2-sulfonyl)-azepan-4-ylcarbamoyl]-cyclohexyl}-amide.

### Process for Synthesis of Inventive Compounds

Referring to Schemes 1-11 herein above, the present invention provides a process for the synthesis of compounds of Formula (I) comprising the step of oxidizing the appropriate compound of Formula (II) with an oxidant to provide the compound of Formula (I) as a mixture of diastereomers. Preferably the oxidant is Dess-Martin periodinane or pyridine sulfur trioxide complex in DMSO and triethylamine.

Referring to Scheme 8, the present invention also provides a process for the synthesis of deuterated compounds of Formula (I). Specifically, when a deuterated isomer is desired, an additional step, following the oxidation step, of deuterating the protonated isomer with a deuterating agent to provide the deuterated compound of Formula (I) as a mixture of diastereomers is added to the synthesis. Preferably, the deuterating agent is CD₃OD:D₂O (10:1) in triethylamine.

The process further comprises the step of separating the diasteromers of Formula (I) by separating means, preferably by high presssure liquid chromatography (HPLC).

### Utility of the Present Invention

The compounds of Formula I are useful as protease inhibitors, particularly as inhibitors of cysteine and serine proteases, more particularly as inhibitors of cysteine proteases, even more particularly as inhibitors of cysteine proteases of the papain superfamily, yet more particularly as inhibitors of cysteine proteases of the cathepsin family, most particularly as inhibitors of cathepsin K. The present invention also provides useful compositions and formulations of said compounds, including pharmaceutical compositions and formulations of said compounds.

The present compounds, especially the preferred 1-amino-1-acyl cyclohexane compounds exhibit superior selectivity for cathepsin K versus cathepsins L,S, and B when compared to structurally similar compounds containing a leucine instead of 1-amino-1-acyl cyclohexane.

The present compounds are useful for treating diseases in which cysteine proteases are implicated, including infections by pneumocystis carinii, trypsanoma cruzi, trypsanoma brucei, and Crithidia fusiculata; as well as in schistosomiasis, malaria, tumor metastasis, metachromatic leukodystrophy, muscular dystrophy, amytrophy; and especially diseases in which cathepsin K is implicated, most particularly diseases of excessive bone or cartilage loss, including osteoporosis, gingival disease including gingivitis and periodontitis, arthritis, more specifically, osteoarthritis and rheumatoid arthritis, Paget's disease; hypercalcemia of malignancy, and metabolic bone disease.

Metastatic neoplastic cells also typically express high levels of proteolytic enzymes that degrade the surrounding matrix, and certain tumors and metastatic neoplasias may be effectively treated with the compounds of this invention.

The present invention also provides methods of treatment of diseases caused by pathological levels of proteases, particularly cysteine and serine proteases, more particularly cysteine proteases, even more particularly cysteine proteases of the papain superfamily, yet more particularly cysteine proteases of the cathepsin family, which methods comprise administering to an animal, particularly a mammal, most particularly a human in need thereof a compound of the present invention. The present invention especially provides methods of treatment of diseases caused by pathological levels of cathepsin K, which methods comprise administering to an animal, particularly a mammal, most particularly a human in need thereof an inhibitor of cathepsin K, including a compound of the present invention. The present invention particularly provides methods for treating diseases in which cysteine proteases are implicated, including infections by pneumocystis carinii, trypsanoma cruzi, trypsanoma brucei, and Crithidia fusiculata; as well as in schistosomiasis, malaria, tumor metastasis, metachromatic leukodystrophy, muscular dystrophy, amytrophy, and especially diseases in which cathepsin K is implicated, most particularly diseases of excessive bone or cartilage loss, including osteoporosis, gingival disease including gingivitis and periodontitis, arthritis, more specifically, osteoarthritis and rheumatoid arthritis, Paget's disease, hypercalcemia of malignancy, and metabolic bone disease.

This invention further provides a method for treating osteoporosis or inhibiting bone loss which comprises internal administration to a patient of an effective amount of a compound of Formula I, alone or in combination with other inhibitors of bone resorption, such as bisphosphonates (i.e., allendronate), hormone replacement therapy, anti-estrogens, or calcitonin. In addition, treatment with a compound of this invention and an anabolic agent, such as bone morphogenic protein, iproflavone, may be used to prevent bone loss or to increase bone mass.

For acute therapy, parenteral administration of a compound of Formula I is preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg, in a manner to maintain the concentration of drug in the plasma at a concentration effective to inhibit cathepsin K. The compounds are administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect.

The compounds of this invention may also be administered orally to the patient, in a manner such that the concentration of drug is sufficient to inhibit bone resorption or to achieve any other therapeutic indication as disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

No unacceptable toxicological effects are expected when compounds of the present invention are administered in accordance with the present invention.

### Biological Assays

The compounds of this invention may be tested in one of several biological assays to determine the concentration of compound which is required to have a given pharmacological effect.

Determination of Cathepsin K Proteolytic Catalytic Activity

All assays for cathepsin K were carried out with human recombinant enzyme. Standard assay conditions for the determination of kinetic constants used a fluorogenic peptide substrate, typically Cbz-Phe-Arg-AMC, and were determined in 100 nM Na acetate at pH 5.5 containing 20 mM cysteine and 5 mM EDTA. Stock substrate solutions were prepared at concentrations of 10 or 20 mM in DMSO with 20 uM final substrate concentration in the assays. All assays contained 10% DMSO. Independent experiments found that this level of DMSO had no effect on enzyme activity or kinetic constants. All assays were conducted at ambient temperature. Product fluorescence (excitation at 360 nM; emission at 460 nM) was monitored with a Perceptive Biosystems Cytofluor II fluorescent plate reader. Product progress curves were generated over 20 to 30 minutes following formation of AMC product.

Inhibition Studies

Potential inhibitors were evaluated using the progress curve method. Assays were carried out in the presence of variable concentrations of test compound. Reactions were initiated by addition of enzyme to buffered solutions of inhibitor and substrate. Data analysis was conducted according to one of two procedures depending on the appearance of the progress curves in the presence of inhibitors. For those-compounds whose progress curves were linear, apparent inhibition constants (K_{i,app}) were calculated according to equation 1 (Brandt et al., Biochemitsry, 1989, 28, 140):

v=VₘA/[Kₐ(1+1/K_{i, app})+A] (1)

where v is the velocity of the reaction with maximal velocity Vₘ, A is the concentration of substrate with Michaelis constant of Kₐ, and I is the concentration of inhibitor.

For those compounds whose progress curves showed downward curvature characteristic of time-dependent inhibition, the data from individual sets was analyzed to give kobs according to equation 2:

[AMC]=vₛₛt+(v₀−vₛₛ)[1−exp(−k_{obs}t)]]/k_{obs} (2)

where [AMC] is the concentration of product formed over time t, v₀ is the initial reaction velocity and vₛₛ is the final steady state rate. Values for k_{obs} were then analyzed as a linear function of inhibitor concentration to generate an apparent second order rate constant (k_{obs}/inhibitor concentration or k_{obs}/[I]) describing the time-dependent inhibition. A complete discussion of this kinetic treatment has been fully described (Morrison et al., Adv. Enzymol. Relat. Areas Mol. Biol., 1988, 61, 201).

This assay measures the affinity of inhibitors to cysteine proteases, in this case, especially cathepsin K, as well as cathepsins L,S, and B. The skilled artisan would consider any compound exhibiting a Kᵢ value of less than 1 micromolar to be a potential lead compound for further research, and an inhibitor exhibiting a Kᵢ of less than 100 nM to be a drug development drug candidate assuming an acceptable pathology/toxicology profile and in vivo activity.

Table I, below, provides inhibition assay data for selected compounds of the present invention. These data demonstrate that the inventive compounds efficiently inhibit cathepsin K. These data also show that the present compounds are highly selective for binding with cathepsin K, compared to binding with cathepsins L, S, or B.

Human Osteoclast Resorption Assay

Aliquots of osteoclastoma-derived cell suspensions were removed from liquid nitrogen storage, warmed rapidly at 37° C. and washed x1 in RPMI-1640 medium by centrifugation (1000 rpm, 5 min at 4° C.). The medium was aspirated and replaced with urine anti-HLA-DR antibody, diluted 1:3 in RPMI-1640 medium, and incubated for 30 min on ice The cell suspension was mixed frequently.

The cells were washed x2 with cold RPMI-1640 by centrifugation (1000 rpm, 5 in at 4° C.) and then transferred to a sterile 15 mL centrifuge tube. The number of mononuclear cells were enumerated in an improved Neubauer counting chamber.

Sufficient magnetic beads (5/mononuclear cell), coated with goat anti-mouse IgG, were removed from their stock bottle and placed into 5 mL of fresh medium (this washes away the toxic azide preservative). The medium was removed by immobilizing the beads on a magnet and is replaced with fresh medium.

The beads were mixed with the cells and the suspension was incubated for 30 min on ice. The suspension was mixed frequently. The bead-coated cells were immobilized on a magnet and the remaining cells (osteoclast-rich fraction) were decanted into a sterile 50 mL centrifuge tube. Fresh medium was added to the bead-coated cells to dislodge any trapped osteoclasts. This wash process was repeated x10. The bead-coated cells were discarded.

The osteoclasts were enumerated in a counting chamber, using a large-bore disposable plastic pasteur pipette to charge the chamber with the sample. The cells were pelleted by centrifugation and the density of osteoclasts adjusted to 1.5×10⁴/mL in EMEM medium, supplemented with 10% fetal calf serum and 1.7 g/litre of sodium bicarbonate. 3 mL aliquots of the cell suspension (per treatment) were decanted into 15 mL centrifuge tubes. These cells were pelleted by centrifugation. To each tube 3 mL of the appropriate treatment was added (diluted to 50 uM in the EMEM medium). Also included were appropriate vehicle controls, a positive control (87MEM1 diluted to 100 ug/mL) and an isotype control (IgG2 a diluted to 100 ug/mL). The tubes were incubate at 37° C. for 30 min.

0.5 mL aliquots of the cells were seeded onto sterile dentine slices in a 48-well plate and incubated at 37° C. for 2 h. Each treatment was screened in quadruplicate. The slices were washed in six changes of warm PBS (10 mL/well in a 6-well plate) and then placed into fresh treatment or control and incubated at 37° C. for 48 h. The slices were then washed in phosphate buffered saline and fixed in 2% glutaraldehyde (in 0.2M sodium cacodylate) for 5 min., following which they were washed in water and incubated in buffer for 5 min at 37° C. The slices were then washed in cold water and incubated in cold acetate buffer/fast red garnet for 5 min at 4° C. Excess buffer was aspirated, and the slices were air dried following a wash in water.

The TRAP positive osteoclasts were enumerated by bright-field microscopy and were then removed from the surface of the dentine by sonication. Pit volumes were determined using the Nikon/Lasertec ILM21W confocal microscope.

TABLE 1
Assay Data

| | |
|---|---|
| 1) | Benzofuran-2-carboxylic acid {1-[(S)-3-oxo-1-(pyridine-2-sulfonyl)- |
| | azepan-4-ylcarbamoyl]-cyclohexyl}-amide (Example 1): |
| | Ki = 1.4 nM (human Cathepsin K) |
| | Ki = 239 nM (human Cathepsin L) |
| | Ki = 390 nM (human Cathepsin S) |
| | Ki = 926 nM (human Cathepsin B) |
| 2) | Benzofuran-2-carboxylic acid {1-[(R)-3-oxo-1-(pyridine-2-sulfonyl)- |
| | azepan-4-ylcarbamoyl]-cyclohexyl}-amide (See Example 1): |
| | Ki = >26 nM (human Cathepsin K), sample may not have been |
| | completely free of S isomer. |
| 3) | Thieno[3,2-b]thiophene-2-carboxylic acid {1-[(S)-3-oxo-1- |
| | (pyridine-2-sulfonyl)-azepan-4-ylcarbamoyl]-cyclohexyl}-amide |
| | (Example 2): |
| | Ki = 0.58 nM (human Cathepsin K) |
| | Ki = 270 nM (human Cathepsin L) |
| | Ki = 632 nM (human Cathepsin S) |
| | Ki = 434 nM (limnan Cathepsin B) |
| 4) | Furo[3,2-b]pyridine-2-carboxylic acid {(S)-3-methyl-1-[(4S,7R)-7 |
| | methyl-3-oxo-1-(pyridine-2-sulfonyl)-azepan-4-ylcarbamoyl]-butyl}- |
| | amide (Example 3): |
| | Ki = 1.8 nM (human Cathepsin K) |
| | Ki = 124 nM (human Cathepsin L) |
| | Ki = 317 nM (human Cathepsin S) |
| | Ki = 198 nM (human Cathepsin B) |
| 5) | Thieno[3,2-b]thiophene-2-carboxylic acid {1-[(4S,7R)-7-methyl-3- |
| | oxo-1-(pyridine-2-sulfonyl)-azepan-4-ylcarbamoyl]-cyclohexyl}- |
| | amide (Example 5): |
| | Ki = 0.3 nM (human Cathepsin K) |
| | Ki = 69 nM (human Cathepsin L) |
| | Ki = 175 nM (human Cathepsin S) |
| | Ki = 173 nivI (human Cathepsin B) |

### General

Nuclear magnetic resonance spectra were recorded at either 250 or 400 MHz using, respectively, a Bruker AM 250 or Bruker AC 400 spectrometer. CDCl₃ is deuteriochloroform, DMSO-d₆ is hexadeuteriodimethylsulfoxide, and CD₃OD is tetradeuteriomethanol. Chemical shifts are reported in parts per million (d) downfield from the internal standard tetramethylsilane. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. Continuous wave infrared (IR) spectra were recorded on a Perkin-Elmer 683 infrared spectrometer, and Fourier transform infrared (PTIR) spectra were recorded on a Nicolet Impact 400 D infrared spectrometer. IR and FTIR spectra were recorded in transmission mode, and band positions are reported in inverse wavenumbers (cm⁻¹). Mass spectra were taken on either VG 70 FE, PE Syx API III, or VG ZAB HF instruments, using fast atom bombardment (FAB) or electrospray (ES) ionization techniques. Elemental analyses were obtained using a Perkin-Elmer 240C elemental analyzer. Melting points were taken on a Thomas-Hoover melting point apparatus and are uncorrected. All temperatures are reported in degrees Celsius.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel.

Where indicated, certain of the materials were purchased from the Aldrich Chemical Co., Milwaukee, Wis., Chemical Dynamics Corp., South Plainfield, N.J., and Advanced Chemtech, Louisville, Ky.

### EXAMPLES

In the following synthetic examples, temperature is in degrees Centigrade (° C.). Unless otherwise indicated, all of the starting materials were obtained from commercial sources. Without further elaboration, it is believed that one skilled in the art can, using the preceding description, utilize the present invention to its fullest extent. These Examples are given to illustrate the invention, not to limit its scope. Reference is made to the claims for what is reserved to the inventors hereunder.

### Example 1

Preparation of Benzofaran-2-carboxylic Acid {1-[(S)-3-oxo-1-(pyridine-2-sulfonel)-azepan-4-ylcarbamoyl]-cyclohexyl}-amide

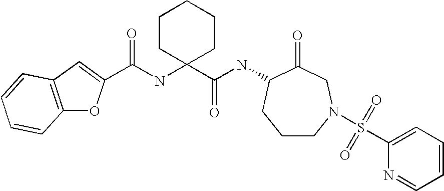

a.) Alkyl-pent-4-enyl-carbamic Acid Benzyl Ester

To a suspension of NaH (1.83 g, 76.33 mmol of 90% NaH) in DMF was added benzyl alkyl-carbamic acid benzyl ester (7.3 g, 38.2 mmol) in a dropwise fashion. The mixture was stirred at room temperature for approximately 10 minutes whereupon 5-bromo-1-pentene (6.78 mL, 57.24 mmol) was added in a dropwise fashion. The reaction was heated to 40° C. for approximately 4 hours whereupon the reaction was partitioned between dichloromethane and water. The organic layer was washed with water (2x's), brine, dried (MgSO₄), filtered and concentrated. Column chromatography of the residue (10% ethyl acetate:hexanes) provided 10.3 grams of the title compound as an oil: MS(EI) 260 (M+H⁺).

b.) 2,3,4,7-Tetrahydro-azepine-1-carboxylic Acid Benzyl Ester

To a solution of alkyl-pentenyl-carbamic acid benzyl ester (50 g) in dichloromethane was added bis(tricyclohexylphosphine)benzylidine ruthenium (V) dichloride (5.0 g). The reaction was heated to reflux until complete as determined by TLC analysis. The reaction was concentrated int vacuo. Column chromatography of the residue (50% dichloromethane:hexanes) gave 35 g of the title compound: MS(I) 232 (M+H⁺).

c.) 8-Oxa-3-aza-bicyclo[5.1.0]octane-3-carboxylic Acid Benzyl Ester

To the solution of 2,3,4,7-Tetrahydro-azepine-1-carboxylic acid benzyl ester (13 g, 56.3 mmol) in CH₂Cl₂ (500 ml) was added NaHCO₃ (14.2 g, 169 mmol) and then mCPBA (29 g, 169 mmol) in portions. Stirred at RT for 4 h before worked up by washing with 15% NaOH, saturated K₂CO₃ and brine. Dried over Na₂SO₄. The reaction mixture was concentrated by rotary evaporation. the title compound was prepared: MS(EI) 248 (M+H⁺), 270(M+Na⁺).

d.) 4-Azido-3-hydroxy-azepane-1-carboxylic Acid Benzyl Ester

To a solution of 8-oxa-3-aza-bicyclo[5.1.0]octane-3-carboxylic acid benzyl ester (2.0 g, 8.1 mmol) in methanol:water (8:1 solution) was added NH₄Cl (1.29 g, 24.3 mmol) and sodium azide (1.58 g, 24.30 mmol). The reaction was heated to 40° C. until complete consumption of the starting epoxide was observed by TLC analysis. The majority of the solvent was removed in vacuo and the remaining solution was partitioned between ethyl acetate and pH 4 buffer. The organic layer was washed with sat. NaHCO₃, water, brine dried (MgSO₄), filtered and concentrated. Column chromatography (20% ethyl acetate:hexanes) of the residue provided 1.3 g of the title compound: MS(EI) 291 (M+H⁺) plus 0.14 g of trans-4-hydroxy-3-azido-hexahydro-1H-azepine

e.) 4-amino-3-hydroxy-azepane-1-carboxylic Acid Benzyl Ester

To a solution of 4-azido-3-hydroxy-azepane-1-carboxylic acid benzyl ester (1.1 g, 3.79 mmol) in methanol was added triethylamine (1.5 mL, 11.37 mmol) and 1,3-propanedithiol (1.1 mL, 11.37 mL). The reaction was stirred until complete consumption of the starting material was observed by TLC analysis whereupon the reaction was concentrated in vacuo. Column chromatography of the residue (20% methanol:dichloromethane) provided 0.72 g of the title compound: MS (EI) 265 (M+H⁺).

f.) 4-{(1-(1-tert-Butoxycarbonylamino-cyclohexyl)-methanoyl]-amino}-3-hydroxy-azepane-1-carboxylic Acid Benzyl Ester

To a solution of the amino alcohol of 4-amino-3-hydroxy-azepane-1-carboxylic acid benzyl ester (1.17 g, 3.89 mmol) in 10 ml DMP was added HBTU (1.47 g, 3.89 mmol), NMM(1.57 g, 15.6 mmol) and Boc-1-amino-1-cyclohexane carboxylic acid(0.95 g, 3.89 mmol). The reaction was stirred at room temperature overnight. The solvent was removed in vacuo. The residue was diluted with ethyl acetate and washed with sat. NaHCO₃, water, brine, dried (MgSO₄), filtered and concentrated to give 2.0 g of the title compound: MS(EI) 490A (M+H⁺).

g.) [1-(3-Hydroxy-azepan-4-ylcarbamoyl)-cyclohexyl]-carbamic Acid Tert-Butyl Ester

To a solution of 4{[1-(1-tert-butoxycarbonylamnuo-cyclohexyl)-methanoyl]-amino}-3-hydroxy-azepane-1-carboxylic acid benzyl ester (1.6 g, 3.27 mmol) and 10% Pd/C (0.4 g, catalytic) in ethanol was affixed a balloon of hydrogen. The reaction was stirred until complete consumption of the starting material was observed by TLC analysis. The reaction was filtered to remove the catalyst and the filtrate was concentrated in vacuo to provide 1.0 g of the title compound: MS(EI) 356.4 (M+H⁺).

h.) {1-[3-Hydroxy-1-(pyridine-2-sulfonyl)-azepan-4-ylcarbamoyl]-cyclohexyl}-carbamic Acid tert-butyl Ester

To a solution of the compound of [1-(3-hydroxy-azepan-4-ylcarbamoyl)-cyclohexyl]-carbamic acid tert-butyl ester (0.45 g, 1.27 mmol) in CH₂Cl₂ was added pyridosulphonylchloride (0.23 g, 1.27 mmol) followed by NNW (0.26 g, 2.54 mmol). The reaction was stirred at room temperature overnight. The mixture was diluted with CH₂Cl₂ washed with sat. NaHCO₃, water, brine, dried (Na₂SO₄), filtered and concentrated. Column chromatography of the residue (2% methanol:dichloromethane) gave 0.44 g of the title compound: MS(EI) 497.4 (M+H⁺).

i.) 1-Amino-cyclohexanecarboxylic Acid [3-hydroxy-1-(pyridine-2-sulfonyl)-azepan-4-yl]-amide

To a solution of the compound of {1-[3-hydroxy-1-(pyridine-2-sulfonyl)-azepan-4-ylcarbamoyl]-cyclohexyl}-carbamic acid tert-butyl ester (0.44 g, 0.89 mmol) in 1.4-dioxan (3 mL) was added 4M HCl in dioxane (4.4 mL). The reaction was stirred at room temperature for 2 hr whereupon it was concentrated in vacuo to give 420 mg of the title compound: MS(ES) 397.2(M+H+).

j.) Benzofuran-2-carboxylic Acid {1-[3-hydroxy-1-(pyridine-2-sulfonyl)-azepan-4-ylcarbamoyl]-cyclohexyl}-amide

To a solution of the amine salt of 1-amino-cyclohexanecarboxylic acid [3-hydroxy-1-(pyridine-2-sulfonyl)-azepan-4-yl]-amide (420 mg, 0.89 mmol) in DMF was added NMM (0.36 g, 3.56 mmol), HBTU (405 mg, 1.07 mmol) and 2-benzofuran carboxylic acid (173 mg, 1.07 mmol). The reaction was stirred until complete by TLC analysis whereupon solvent was removed in vacuo and the residue was dissolved with ethyl acetate and washed with sat. NaHCO₃, water, brine, dried (Na₂SO₄), filtered and concentrated. Column chromatography of the residue (3% methanol:dichloromethane) gave 400 mg of the title compound: MS(EI) 541.2 (M+H⁺).

k.) Benzofuran-2-carboxylic acid {1-[3-oxo-1-(pyridine-2-sulfonyl)-azepan-4-ylcarbamoyl]-cyclohexyl}-amide

To a solution of benzofuran-2-carboxylic acid {1-[3-hydroxy-1-(pyridine-2-sulfonyl)-azepan-4-ylcarbamnoyl]-cyclohexyl}-amide (200 mg, 0.37 mmol) in 5 ml CH₂Cl₂, was added Dess-Martin reagent (240 mg, 0.56 mmol) at RT. The solution was stirred for 2 h when 50 ml CH₂Cl₂, was added and then washed with 1:1 mixture of 10% NaHCO₃ and 10% Na₂S₂O₃ and brine. Purification by column chromatograghy (2% methanol:dichloromethane) gave the title compound (120 mg, 60% yield): ¹H NMR (CDCL): δ.=8.70-8.69 (d, 1H), 7.98-7.91(m, 2H), 7.71-7.69(d, 1H), 7.58-7.28(m, 6H), 6.74(s, 1H), 5.15-5.10(m, 1H), 4.78-4.73(d, 1H), 4.13-4.09(d, 1H), 3.84-3.79(d, 1H), 2.74-2.68(t, 1H), 2.37-1.37(m,14H); MS(EI) 539.2 (M+H⁺)

Separation of the enantiomers by HPLC provided enantiomer 1: MS (EI) 593.2(M+H⁺), and enantiomer 2: MS (EI) 593.2 (M+H⁺).

### Example 2

Preparation of Thieno[3,2-b]thiophene-2-carboxylic Acid {1-[(+/−)-3-oxo-1-(pyridine-2-sulfonyl)-azepan-4-ylcarbamorl]-cyclohexyl}-amide

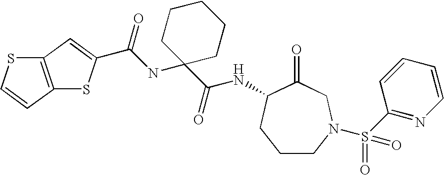

Following the procedure of Example 1, except substituting “thieno[3,2-b]-thiophene-2-carboxylic acid (as described in Kukolja, Stjepan; et al. J. Med. Chem. 1985, 28, 1896-1903)” for “benzofuran-2-carboxylic acid” gives the title compound: ¹H NMR: (DMSO): δ.=8.76-8.74 (d, 1H), 8.30(s, 1H), 8.13-8.11(d, 2H), 8.00-7.98(d, 1H), 7.87-7.86(d, 1H), 7.72(m, 1H), 7.60-7.58(d, 1H), 7.51-7.50(d, 1H), 4.78(m, 1H), 4.45-4.40(d, 1H), 3.91-3.86(d, 2H), 2.87(m, 1H), 2.20(m, 2H), 1.84-1.77(m,5H), 1.56(m, 6M), 1.27(m, 1H); ESMS: (M+H⁺)=561.2

### Example 3

Preparation of Benzofuran-2-carboxylic Acid {1-[(4S,7R)-7-methyl-3-oxo-1-(pyridine-2-sulfonyl)-azepan-4-ylcarbamoyl]-cyclohexl}-amide

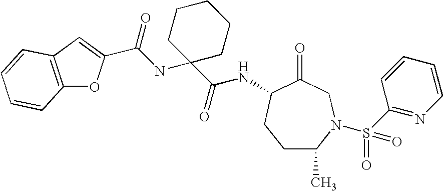

a. ((R)-2-Iodo-1-methyl-ethyl)-carbamic Acid Benzyl Ester

Triphenylphospine (24 g, 91.8 mmol) was added to a solution of imidazole (12.5 g, 184 mmol) in CH₂Cl₂ (231 ml), then was cooled to 0 degrees C. Iodine (23.3 g, 91.8 mmol) was added to the suspension. The reaction mixture turned yellow, then faintly brown. After 5 minutes ((R)-2-hydroxy-1-methyl-ethyl)-carbamic acid benzyl ester (9.59 g, 45.9 mmol) was added and the reaction mixture was warmed to RT then stirred for 3 h. Then, H₂O (7 ml) was added and the reaction mixture was partitioned between CH₂Cl₂ (300 ml) and H₂O (600 ml). The aqueous layer was extracted again with CH₂Cl₂ (200 ml). The combined organic layer was then washed with a solution of 1:9 aq. saturated Na₂S₂O₃: H₂O (140 ml), then brine (400 ml). The combined organics were dried with MgSO₄, filtered, concentrated in vacuo, then filtered through a plug of silica gel washing with 15% EtOAc/hexanes (1.5 liter). The solution was concentrated in vacuo, then the solid was washed with hexane and the resultant white solid was used in the next reaction without further purification (11 g, 75%).

b. ((R)-1-Methyl-pent-4-enyl)-carbamic Acid Benzyl Ester

Copper (I) bromide-dimethyl sulfide (1.93 g, 9.4 mmol) was dissolved in distilled THF (24 ml), then was cooled to −78 degrees C. A solution of alkyl magnesium chloride (9.4 ml, 2M in THF, Aldrich) was added dropwise, then the solution was stirred for 30 minutes. ((R)-2-Iodo-1-methyl-ethyl)arbamic acid benzyl ester (1.5 g, 4.7 mmol) in distilled THF (3 ml) was added dropwise, then the reaction was warmed to −40 degrees C. and was stirred for 2.5 h. The reaction mixture was quenched with aq. sat. NH₄Cl (4 ml) at −40 degrees C., warmed to RT and the gray reaction mixture turned sky blue. THF was removed in vacuo. Then, Et₂O was added and the reaction mixture was filtered to remove precipitated solids. The solids were washed with additional Et₂O. The combined organics were extracted with 10% NH₄OH (3×), then brine. The combined organics were dried with MgSO₄, filtered, concentrated in vacuo, then filtered through a plug of silica gel washing with 20% EtOAc/hexanes (100 ml). The solution was concentrated in vacuo, then the resultant colorless oil was used in the next reaction without further purification (0.8 g, 73%).

c. Alkyl-((R)-1-methyl-pent-4-enyl)-carbamic Acid Benzyl Ester

((R)-1-Methyl-pent-4-enyl)-carbamic acid benzyl ester (790 mg, 3.39 mmol) was dissolved in DMF (8 ml) and was cooled to 0 degrees C. Sodium hydride (60% dispersion, 271 mg, 6.78 mmol) was added and the reaction was stirred for 15 minutes. Alkyl bromide (1.23 g, 0.88 ml, 10.17 mmol) was added and the reaction mixture was stirred for 3 h at 0 degrees C. H₂O (10 ml) was added, then 2N HCl was added dropwise adjusting the pH to 1. The reaction mixture was extracted with Et20 (2×50 ml). The combined organics were washed with aq. 2N HCl, then aq. NaHCO₃, then brine. The combined organics were dried with MgSO₄, filtered, concentrated in vacuo, then chromatographed on silica gel (5% EtOAc/hexanes) to yield the title compound as a colorless oil (883 mg, 95%).

d. 2-Methyl-2,3,4,7-tetrahydro-azepine-1-carboxylic Acid Benzyl Ester

Alkyl-(1-methyl-pent-4-enyl)-carbamic acid benzyl ester (0.872 g, 3.19 mmol) was dissolved in CH₂Cl₂ (10 ml) and a stream of argon gas was bubbled into the reaction mixture for 10 miinutes. Then bis(tricyclohexylphosphine)benzylidine ruthenium(IV) dichloride (Strem Chemicals, Grubbs' catalyst, 19 mg, 0.0227 mmol) was added and the reaction mixture was refluxed for 2 h. Additional bis(tricyclohexylphosphine)benzylidine ruthenium(IV) dichloride (mg, 0.0108 mmol) was added and the reaction mixture was refluxed for an additional 1.5 hours. The reaction was cooled to RT under argon overnight, then was concentrated in vacuo by rotary evaporation, then was chromatographed (silica gel, 5% EtOAc/hexanes) to give the title compound (0.72 g, 92%): ¹H NMR: 7.35-7.20 (m, 5H), 5.65 (1H, m), 5.13 (2H, AB), 4.454.05 (m, 2H), 3.56 (1H, d), 2.25-2.10 (m, 2H), 1.90-1.60 (m, 2H), 1.12 (3H, d); Liquid Chromatgraphy/Electrospray mass spec: M+H⁺=246.2.

e. (1S,4R,7R)₄Methyl-8-oxa-3-aza-bicyclo[5.1.0]octane-3-carboxylic Acid Benzyl Ester

m-Chloro-perbenzoic acid (1.10 g, 57-86% pure) was added to a solution of 2-methyl-2,3,4,7-tetrahydro-azepine-1-carboxylic acid benzyl ester (0.72 g, 2.94 mmol) in CH₂Cl₂ at 0 degrees C. The reaction mixture was stirred for half an hour, then was warmed to RT. Additional m-chloro-perbenzoic acid (0.660 g, 57-86% pure) was added and the reaction was stirred 2 h. The reaction mixture was concentrated in vacuo by rotary evaporation, then 80 ml of 9:1 hexanes/EtOAc was added and the reaction mixture was filtered. The filtrate was concentrated in vacuo by rotary evaporation, then was chromatographed (silica gel, 20% EtOAc:hexanes) to give (1S,4R,7S)-4-methyl-8-oxa-3-aza-bicyclo[5.1.0]octane-3-carboxylic acid benzyl ester (0.450 g, 75%) and the title compound (0.15 g, 25% yield): ¹H NMR: 7.42-7.22 (m, 5H), 5.13 (2H, s), 4.50-4.15 (m, 2H), 3.27 (1H, d), 3.12-2.95 (1H, m), 2.15-1.70 (m, 2H), 1.47 (m, 2H), 1.12 (3H, d); Liquid Chromatgraphy/Electrospray mass spec: M+H⁺=262.0.

f. (2R,5S,6S)-5-Azido-6-hydroxy-2-methyl-azepane-1-carboxylic Acid Benzyl Ester

Sodium azide (0.139 g, 2.14 mmol) was added to a solution of (1S,4R,7R)-4-methyl-8-oxa-3-aza-bicyclo[5.1.0]octane-3-carboxylic acid benzyl ester (0.186 g, 0.71 mmol) and ammonium chloride (0.114 g, 2.14 mmol) in MeOH (1.5 ml) and H₂O (0.15 ml), then was refluxed for 6 h. The reaction mixture was concentrated in vacuo by rotary evaporation, then was diluted with water (5 ml) and extracted with EtOAc (10 ml). The organic layer was then extracted with water, brine, dried with MgSO₄, filtered, concentrated in vacuo by rotary evaporation, and chromatographed (silica gel, 20% EtOAc/hexanes) to yield the title compound (0.192 g, 89%): 7.39-7.30 (m, 5H), 5.15 (2H, s), 4.10-3.67 (m, 2H), 3.10 (1H, d), 1.85-1.53 (m, 4H), 1.09 (3H, d); Liquid Chromatgraphy/Electrospray mass spec: M+H⁺=305.2.

g. (2R,5S,6S)-5-Amino-6-hydroxy-2-methyl-azepane-1-carboxylic Acid Benzyl Ester

Triphenylphosphine (0.25 g, 0.952 mmol) was added to a solution of (2R,5S,6S)-5-azido-6-hydroxy-2-methyl-azepane-1-carboxylic acid benzyl ester (0.193 g, 0.635 mmol) in THF (10 ml) and H₂O (0.04 ml), then was heated to 45 degrees C. overnight. The reaction mixture was then diluted with toluene (100 ml×2) and was azeotroped iii vacuo by rotary evaporation twice. The resulting oil was dissolved in MeOH and HCl in Et₂O and the resulting salt was collected following filtration and was used in the next reaction without further purification (0.27 g, 90%).

h. (2R,5S,6S)-5-{[1-(1-tert-Butoxycarbonylaminoyclohexyl)-methanoyl]-amino)}-6-hydroxy-2-methyl-azepane-1-carboxylic Acid Benzyl Ester

4-methylmorpholine (388 mg, 0.42 ml, 3.84 mmol) was added to a solution of Boc-1-amino-cyclohexanecarboxylic acid (255 mg, 1.05 mmol), HBTU(398 mg, 1.05 mmol), and (2R,5S,6S)-5-Amino-6-hydroxy-2-methyl-azepane-1-carboxylic acid benzyl ester (300 mg, 0.96 mmol) in DMF (5.0 ml). The reaction was stirred overnight at RT, then was diluted with EtOAc (100 ml) after the removal of DMF, washed with NaHCO₃(50 ml), brine (50 ml), dried with magnesium sulfate, filtered, concentrated in vacuo by rotary evaporation, and chromatographed (silica gel, 50% EtOAc/hexanes) to yield the title compound (320 mg, 66%): Electrospray mass spec: M+H⁺=504.2.

i. [1-((3S,4S,7R)-3-Hydroxy-7-methyl-azepan-4-ylcarbamoyl)-cyclohexyl]-carbamic Acid-tert-butyl Ester

(2R,5S,6S)-5-{[1-(1-tert-Butoxycarbonylamino-cyclohexyl)-methanoyl]-amino}-6-hydroxy-2-methyl-azepane-1-carboxylic acid benzyl ester (320 mg, 0.636 mmol) was dissovled in EtOH (10 ml). Then 10% Pd/C (0.1 g) was added and the reaction was stirred for 5 h under a balloon filled with hydrogen gas. The reaction mixture was filtered through Celite, concentrated in vacuo by rotary evaporation and was used in the next reaction without further purification (0.24 g): Electrospray mass spec: M+H⁺=370.2

j. {1-[(3S,4S,7R)-3-Hydroxy-7-methyl-1-(pyridine-2-sulfonyl)-azepan-4-ylcarbamoyl]-cyclohexyl}-carbamic Acid-tert-butyl Ester

2-Pyridine sulfonyl chloride (114 mg, 0.64 mmol) was added to a solution [1-((3S,4S,7R)-3-Hydroxy-7-methyl-azepanfylcarbamoyl)-cyclohexyl]-carbamic acid-tert-butyl ester (240 mg, 0.64 mmol), Triethylamine (194 mg, 1.92 mmol) in CH₂Cl₂ (5 ml) and was stirred at RT over night. The reaction mixture was diluted with CH₂Cl₂ (100 ml), washed with NaHCO₃, brine, dried with magnesium sulfate, filtered, concentrated in vacuo by rotary evaporation, and chromatographed (silica gel, 50% to 75% EtOAc/hexanes) to yield the title compound (0.2 g, 61%): Electrospray mass spec: M+H⁺=511.2

k. 1-Amino-cyclohexanecarboxylic Acid [(3S,4S,7R)-3-hydroxy-7-methyl-1-(pyridine-2-sulfonyl)-azepan-4-yl]-amide

HCl in dioxane (4.0 M, 5.0 ml) was added to a stirred solution {1-[(3S,4S,7R)-3-Hydroxy-7-methyl-1-(pyridine-2-sulfonyl)-azepan-4-ylcarbamoyl]-cyclohexyl}-carbamic acid-tert-butyl ester (200 mg, 0.39 mmol) in MeOH (1.0 ml). The reaction mixture was stirred for 2 h at RT, then was concentrated in vacuo by rotary evaporation and was used in the next reaction without further purification (190 mg). M+H⁺=411.4

l. Benzofuran-2-carboxylic acid {1-[(3S,4S,7R)-3-hydroxy-7-methyl-1-(pyridine-2-sulfonyl)-azepan-4-ylcarbamoyl]-cyclohexyl}-amide

4-methylmorpholine (81 mg, 0.8 mmol) was added to a solution of benzofuran-2-carboxylic acid (38.4 mg, 0.24 mmol), HBTU(91 mg, 0.24 mmol), and 1-Amino-cyclohexanecarboxylic acid [(3S,4S,7R)-3-hydroxy-7-methyl-1-(pyridine-2-sulfonyl)-azepan-4-yl]-amide (95 mg, 0.2 mmol) in DMF (5.0 ml). The reaction was stirred overnight at RT, then was diluted with EtOAc (100 ml) after the removal of DMF, washed with NaHCO₃(50 ml), brine (50 ml), dried with magnesium sulfate, filtered, concentrated in vacuo by rotary evaporation, and chromatographed (silica gel, 2.0% MeOH/CH₂Cl₂) to yield the title compound (90 g, 81%): Electrospray mass spec: M+H⁺=555.4

m. Benzofuran-2-carboxylic Acid {1-[(4S,7R)-7-methyl-3-oxo-1-(pyridine-2-sulfonyl)-azepan-4-ylcarbamoyl]-cyclohexyl}-amide

Dess-Martin periodinane (137 mg, 0.324 mmol) was added to a solution of Benzofuran-2-carboxylic acid {1-[(3S,4S,7R)-3-hydroxy-7-methyl-1-(pyridine-2-sulfonyl)-azepan-4-ylcarbamoyl]-cyclohexyl}-amide (90 mg, 0.162 mmol) in CH₂Cl₄ (5 ml) and was stirred at RT for 2 h. The solution was washed with 10% aq. Na₂S₂O₃, then aq. sat. NaHCO₃, then brine. Purification by column chromatography (silica gel, 2.0% MeOH/CH₂Cl₂) gave the title compound (58 mg, 64%): ¹H NMR: (CDCl): δ.=8.748.73 (d, 1H), 8.02-7.91(m, 2H), 7.71-7.69(d, 1H), 7.58-7.28(m, 6H), 6.73(s, 1H), 5.10-5.08(m, 1H), 4.78-4.73(d, 1H), 4.44(m, 1H), 3.863.81(d, 1H), 2.33-2.01(m, 6H), 1.98-1.40(m,8H), 0.99-0.97(d, 3H); MS(EI): (M+H⁺)=553.4

### Example 4

Preparation of 2,2,4-Trideutero-Benzofuran-2-carboxylic Acid {1-[(4S,7R)-7-methyl-3-oxo-1-(pyridine-2-sulfonyl)-azepan-4-ylcarbamoyl]-cyclohexyl}-amide

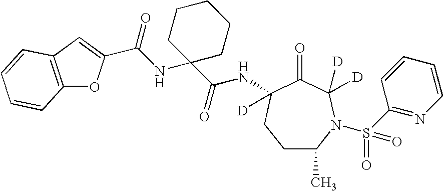

Benzofuran-2-carboxylic acid {1-[(4S,7R)-7-methyl-3-oxo-1-(pyridine-2-sulfonyl)-azepan-4-ylcarbamoyl]-cyclohexyl}-amide is dissolved in d4-methanol (CD₃OD) and D₂O (10:1), then triethyl amine is added and the reaction mixture is stirred for 3 days. Azeotroping with toluene by concentrating iit vacuo provides the title compound.

### Example 5

Preparation of Thieno[3,2-b]thiophene-2-carboxylic Acid {1-[(4S,7R)-7-methyl-3-oxo-1-(pyridine-2-sulfonyl)-azepan-4-ylcarbamoyl]-cyclohexyl}-amide

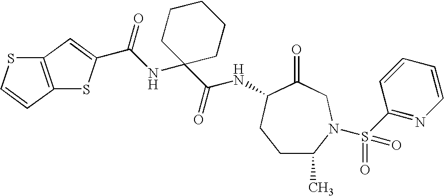

Following the procedure of Example 3, except substituting “thieno[3,2-b]-thiophene-2-carboxylic acid (as described in Kukolja, Stjepan; et al. J. Med. Chem. 1985, 28, 1896-1903)” for “benzofuran-2-carboxylic acid” gives the title compound: ¹H NMR: (CDCl₃) δ.=8.65-8.64 (d, 1H), 7.92-7.85(m, 2H), 7.76(s, 1H), 7.47-7.41(m, 3H), 7.21-7.20(d, 1H), 6.08(s, 1H), 5.01-4.96(m, 1H), 4.69-4.64(d, 1H), 4.35-4.33(m, 1H), 3.77-3.72(d, 1H), 2.25-1.26(m, 14H), 0.90(m, 3H); ESMS: (M+H⁺)=575.2

### Example 6

Preparation of 2,2,4Trideutero-Benzofuran-2-carboxylic Acid {1-[(+/−)-3-oxo-1-(pyridine-2-sulfonyl)-azepan-4-ylcarbamoyl]-cyclohexyl}-amide

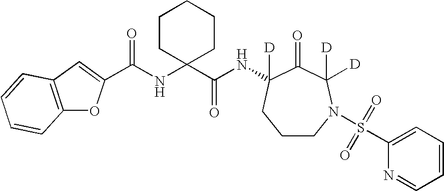

Following the procedure of Example 4, except “benzofuran-2-carboxylic acid {1-[(+/−)-3-oxo-1-(pyridine-2-sulfonyl)-azepan-4-ylcarbamoyl]-cyclohexyl}-amide” for “Benzofaran-2-carboxylic acid {1-[(4S,7R)-7-methyl-3-oxo-1-(pyridine-2-sulfonyl)-azepan-4-ylcarbamoyl]-cyclohexyl}-amide” gives the title compound.

### Example 7

Preparation of 2,2,4Trideutero-Thieno[3,2-b]thiophene-2-carboxylic Acid {1-[(S)-3-oxo-1-(pyridine-2-sulfonyl)-azepan-4-ylcarbamoyl]-cyclohexyl}-amide

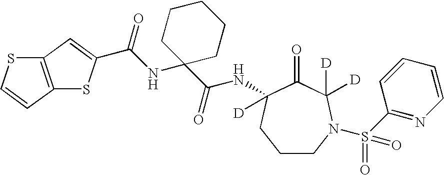

Following the procedure of Example 4, except “thieno[3,2-b]thiophene-2-carboxylic acid {1-[(S)-3-oxo-1-(pyridine-2-sulfonyl)-azepan-4-ylcarbamoyl]-cyclohexyl}-amide” for “Benzofuran-2-carboxylic acid {1-[(4S,7R)-7-methyl-3-oxo-1-(pyridine-2-sulfonyl)-azepan-4-ylcarbamoyl]-cyclohexyl}-amide” gives the title compound.

### Example 8

Preparation of 2,2,4-Trideutero-thieno[3,2-b]thiophene-2-carboxylic Acid {1-[(4S,7R)-7-methyl-3-oxo-1-(pyridine-2-sulfonyl)-azepan-4-ylcarbamoyl]-cyclohexyl}-amide

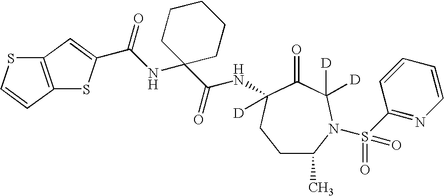

Following the procedure of Example 4, except “thieno[3,2-b]thiophene-2-carboxylic acid {1-[(4S,7R)-7-methyl-3-oxo-1-(pyridine-2-sulfonyl)-azepan-4-ylcarbamoyl]-cyclohexyl}-amide” for “Benzofuran-2-carboxylic acid {1-[(4S,7R)-7-methyl-3-oxo-1-(pyridine-2-sulfonyl)-azepan-4-ylcarbamoyl]-cyclohexyl}-amide” gives the title compound.

The above specification and Examples fully disclose how to make and use the compounds of the present invention. However, the present invention is not limited to the particular embodiments described hereinabove, but includes all modifications thereof within the scope of the following claims. The various references to journals, patents and other publications which are cited herein comprise the state of the art and are incorporated herein by reference as though fully set forth.

We claim:

1. A compound of Formula I: 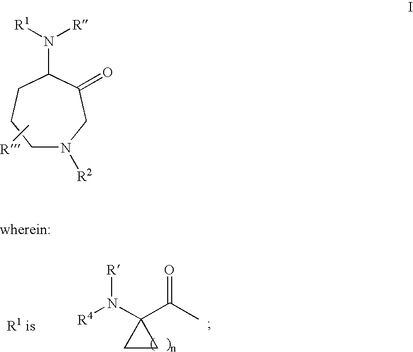
R² is selected from the group consisting of: H, C₁₋₆alkyl, C₃₋₆cycloalkyl-C₀₋₆alkyl, Ar—C₀₋₆alkyl, Het-C₀₋₆alkyl, R⁹C(O)—, R⁹C(S)—, R⁹SO₂—, R⁹OC(O)—, R⁹R¹¹NC(O)—, R⁹R¹¹NC(S)—, R⁹(R¹¹)NSO₂—, 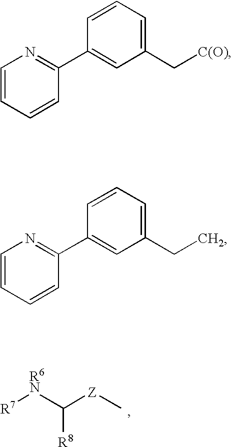
and R⁹SO₂R¹¹NC(O)—;
R⁴ is selected from the group consisting of: H, C₁₋₆alkyl, C₃₋₆cycloalkyl-C₀₋₆alkyl, Ar—C₀₋₆alkyl, Het-C₀₋₆alkyl, R⁵C(O)—, R⁵C(S)—, R⁵SO₂—, R⁵OC(O)—, R⁵R¹²NC(O)—, and R⁵R¹²NC(S)—;
R⁵ is selected from the group consisting of: H, C₁₋₆alkyl, C₂₋₆alkenyl, C₂₋₆alkynyl, C₃₋₆cycloalkyl-C₀₋₆alkyl, C₂₋₆alkanonyl, Ar—C₀₋₆alkyl and Het-C₀₋₆alkyl;
R⁶ is selected from the group consisting of: H, C₁₋₆alkyl, Ar—C₀₋₆alkyl, and Het-C₀₋₆alkyl;
R⁷ is selected from the group consisting of: H, C₁₋₆alkyl, C₃₋₆cycloalkyl-C₀₋₆alkyl, Ar—C₀₋₆alkyl, Het-C₀₋₆alkyl, R¹⁰C(O)—, R¹⁰C(S)—, R¹⁰SO₂—, R¹⁰OC(O)—, R¹¹R¹³NC(O)—, and R¹⁰R¹³NC(S)—;
R⁸ is selected from the group consisting of: H, C₁₋₆alkyl, C₂₋₆alkenyl, C₂₋₆alkynyl, HetC₀₋₆alkyl and ArC₀₋₆alkyl;
R⁹ is selected from the group consisting of: C₁₋₆alkyl, C₃₋₆cycloallkyl-C₀₋₆alkyl, Ar—C₀₋₆alkyl, Ar—COOH, and Het-C₀₋₆alkyl;
R¹⁰ is selected from the group consisting of: C₁₋₆alkyl, C₃₋₆cycloalkyl-C₀₋₆alkyl, Ar—C₀₋₆alkyl and Het-C₀₋₆alkyl;
R¹¹ is selected from the group consisting of: H, C₁₋₆alkyl, Ar—C₀₋₆alkyl, and Het-C₀₋₆alkyl;
R¹² is selected from the group consisting of: H, C₁₋₆alkyl, Ar—C₀₋₆alkyl, and Het-C₀₋₆alkyl;
R¹³ is selected from the group consisting of: H, C₁₋₆alkyl, Ar—C₀₋₆alkyl, and Het-C₀₋₆alkyl;
R′ is selected from the group consisting of: H, C₁₋₆alkyl, Ar—C₀₋₆alkyl, and Het-C₀₋₆alkyl;
R″ is selected from the group consisting of: H, C₁₋₆alkyl, Ar—C₀₋₆alkyl, and Het-C₀₋₆alkyl;
R′″ is selected from the group consisting of: H, C₁₋₆alkyl, C₃₋₆cycloalkyl-C₀₋₆alkyl, Ar—C₀₋₆alkyl, and Het-C₀₋₆alkyl;
Z is selected from the group consisting of: C(O) and CH₂; and
n is an integer from 1 to 5;
and pharmaceutically acceptable salts, hydrates and solvates thereof.

2. A compound according to claim 1 wherein n is 4.

3. A compound according to either claim 1 or 2 wherein R⁴ is selected from the group consisting of: R⁵OC(O)—,R⁵C(O)— or R⁵SO₂—.

4. A compound according to claim 3 wherein R⁴ is R⁵C(O)—.

5. A compound according to claim 4 wherein R⁵ is selected from the group consisting of: C₁₋₆alkyl, C₂₋₆alkenyl, C₃₋₆cycloalkyl-C₀₋₆alkyl, C₂₋₆alkanonyl, Ar—C₀₋₆alkyl and Het-C₀₋₆alkyl.

6. A compound according to claim 5 wherein:
C₁₋₆alkyl is selected from the group consisting of:
methyl, halogenated methyl, C₁₋₆alkoxy and aryloxy substituted methyl,
heterocycle substituted methyl;
ethyl, heterocycle substituted ethyl;
butyl, aryl substituted butyl; and
isopentyl;
C₃₋₆cycloalkyl-C₀₋₆alkyl is cyclohexyl;
C₂₋₆alkenyl is selected from the group consisting of:
butenyl, and aryl substituted butenyl;
C₂₋₆alkanonyl is selected from the group consisting of:
acetyl; and
pentanonyl;
Ar—C₀₋₆alkyl is selected from the group consisting of:
phenyl, phenyl substituted with one or more halogens, phenyl substituted with one or more aryloxy or C₁₋₆alkoxy groups, phenyl substituted with one or more C₁₋₆alkyl sulfonyl groups;
benzyl; and
naphthylenyl; and
Het-C₀₋₆alkyl is selected from the group consisting of:
benzo[1,3]dioxolyl;
furanyl, nitro substituted furanyl, halogen substituted furanyl, aryl substituted furanyl, C₁₋₆alkyl substituted furanyl;
tetrahydrofuranyl;
benzofuranyl, C₁₋₆alkoxy substituted benzofuranyl, halogen substituted benzofuranyl, C₁₋₆alkyl substituted benzofuranyl;
napththo[2,1-b]-furanyl, C₁₋₆alkyl substituted napththo[2,1-b]-furanyl;
benzo[b]thiophenyl, C₁₋₆alkoxy substituted benzo[b]thiophenyl;
quinolinyl;
quinoxalinyl;
1,8 naphthyridinyl;
indolyl, C₁₋₆alkyl substituted indolyl;
pyridinyl, C₁₋₆alkyl substituted pyridinyl, 1-oxy-pyridinyl;
furo[3,2-b]-pyridinyl, C₁₋₆alkyl substituted furo[3,2-b]-pyridinyl;
thiophenyl, C₁₋₆alkyl substituted thiophenyl, halogen substituted thiophenyl;
thieno[3,2-b]thiophenyl C₁₋₆alkyl substituted thieno[3,2-b]thiophen-2-yl;
isoxazolyl, C₁₋₆alkyl substituted isoxazolyl;
oxazolyl, aryl substituted oxazolyl, C₁₋₆alkyl substituted oxazolyl; and
1H-benzoimidazolyl.

7. A compound according to claim 6 wherein:
halogenated methyl is trifluoromethyl;
C₁₋₆alkoxy substituted methyl is selected from the group consisting of: phenoxy-methyl and 4-fluoro-phenoxy-methyl;
heterocycle substituted methyl is 2-thiophenyl-methyl;
heterocycle substituted ethyl is piperidin-1-yl-ethyl;
aryl substituted butyl is 4-(4-methoxy)phenyl-butyl;
pentanonyl is 4-pentanonyl;
aryl substituted butenyl is 4,4-bis(4-methoxyphenyl)-but-3-enyl;
phenyl substituted with one or more halogens is selected from the group consisting of: 3,4-dichlorophenyl and 4-fluorophenyl;
phenyl substituted with one or more aryloxy or C₁₋₆alkoxy groups is selected from the group consisting of: 3,4-dimethoxy-phenyl and 3-benzyloxy-4-methoxy-phenyl;
phenyl substituted with one or more C₁₋₆alkyl sulfonyl groups is 4-methanesulfonyl-phenyl;
naphthylenyl is naphthylen-2-yl;
benzo[1,3]dioxolyl is benzo[1,3]dioxol-5-yl, furanyl is furan-2-yl;
nitro substituted furanyl is 5-nitro-furan-2-yl;
aryl substituted furanyl is selected from the group consisting of: 5-(4-nitrophenyl)-furan-2-yl, 5-(3-triflouromethyl-phenyl)-furan-2-yl, and 5-(4-chloro-phenyl)-furan-2-yl);
halogen substituted furanyl is 5-bromo-furan-2-yl;
C₁₋₆alkyl substituted furanyl is selected from the group consisting of: 3-methyl-furan-2-yl, 4-methyl-furan-2-yl, 2,5-dimethyl-furan-2-yl, and 2,4-dimethyl-furan-2-yl;
tetrahydrofuranyl is tetrahydrofuran-2-yl;
benzofuranyl is benzofuran-2-yl;
C₁₋₆alkoxy substituted benzofuranyl is selected from the group consisting of: 5-(2-piperazin-4-carboxylic acid tert-butyl ester-ethoxy) benzofuran-2-yl, 5-(2-morpholino-4-yl-ethoxy)-benzofuran-2-yl, 5-(2-piperazin-1-yl-ethoxy)benzofuran-2-yl, 5-(2-cyclohexyl-ethoxy)-benzofuran-2-yl, 7-methoxy-benzofuran-2-yl, 5-methoxy-benzofura-2-yl, 5,6-dimethoxy-benzofuran-2-yl5-methoxy-3-methyl-benzofaran-2-yl, 4-methoxy-3-methyl-benzofuran-2-yl, and 6-methoxy-3-methyl-benzofuran-2-yl;
halogen substituted benzofuranyl is selected from the group consisting of: 5-fluoro-benzofuran-2-yl 5,6-difluoro-benzofuran-2-yl5-fluoro-3-methyl-benzofuran-2-yl, and 6-fluoro-3-methyl-benzofuran-2-yl;
C₁₋₆alkyl substituted benzofuranyl is selected from the group consisting of: 3-methyl-benzofuran-2-yl, 3,5-dimethyl-benzofuran-2-yl, and 3-ethyl-benzofuran-2-yl;
napththo[2,1-b]-furanyl is napththo[2,1-b]-furan-2-yl;
C₁₋₆alkyl substituted napththo[2,1-b]-furanyl is 1-methyl-naphtho[2,1-b]-furan-2-yl;
benzo[b]thiophenyl is benzotb]thiophen-2-yl;
C₁₋₆alkoxy substituted benzo[b]thiophenyl is 5,6-dimethoxy-benzo[b]thiophen-2-yl;
quinolinyl is selected from the group consisting of: quinolin-2-yl, quinolin-3-yl, quinolin-4-yl, quinolin-6-yl, and quinolin-8-yl;
quinoxalinyl is quinoxalin-2-yl;
1,8 naphthyridinyl is 1,8 naphthyridin-2-yl;
indolyl is selected from the group consisting of: indol-3-yl and indol-5-yl;
C₁₋₆alkyl substituted indolyl is N-methyl-indol-2-yl;
pyridinyl is selected from the group consisting of: pyridin-2-yl, pyridin-3-yl, and pyridin-5-yl;
1-oxy-pyridinyl is selected from the group consisting of: 1-oxy-pyridin-2-yl and 1-oxy-pyridin-3-yl;
C₁₋₆alkyl substituted pyridinyl is 2-methyl-pyridin-5-yl;
furo[3,2-b]-pyridinyl is furo[3,2-b]-pyridin-2-yl;
C₁₋₆alkyl substituted furo[3,2-b]-pyridinyl is 3-methyl-furo[3,2-b]-pyridin-2-yl;
thiophenyl is thiophen-3-yl;
halogen substituted thiophenyl is 4,5-dibromo-thiophen-2-yl;
C₁₋₆alkyl substituted thiophenyl is 5-methyl-thiophen-2-yl;
thieno[3,2-b]thiophenyl is thieno[3,2-b]thiophene-2-yl;
C₁₋₆alkyl substituted thieno[3,2-b]thiophen-2-yl is 5-tert-butyl-3-methyl thieno[3,2-b]thiophen-2-yl;
isoxazolyl is isoxazolyl;
C₁₋₆alkyl substituted isoxazolyl is 3,5-dimethyl-isoxazol-4-yl;
oxazolyl is oxazolyl;
aryl substituted oxazolyl is 5-methyl-2-phenyl oxazol-4-yl;
C₁₋₆alkyl substituted oxazolyl is 2-phenyl-5-trifluoromethyl-oxazol-4-yl; and
1H-benzoimidazolyl is 1H-benzoimidazol-5-yl.

8. A compound according to claim 7 wherein R⁵ is selected from the group consisting of: benzofuran-2-yl, 3-methyl-benzofuran-2-yl, 5-methoxybenzofuran-2-yl, thieno[3,2-b]thiophen-2-yl, quinoxalin-2-yl, and quinolin-2-yl.

9. A compound according to claim 8 wherein R⁵ is selected from the group consisting of: benzofuran-2-yl and thieno[3,2-b]thiophene-2-yl.

10. A compound according to claim 9 wherein R⁵ is benzofuran-2-yl.

11. A compound according to either claim 1 or 2 wherein R′ is selected from the group consisting of H and naphthalen-2-yl-methyl.

12. A compound according to claim 11 wherein R′ is H.

13. A compound according to either claim 1 or 2 wherein R″ is H.

14. A compound according to either claim 1 or 2 wherein R′″ is selected from the group consisting of H and methyl.

15. A compound according to claim 14 wherein R′″ is methyl.

16. A compound according to either claim 1 or 2 wherein R″ is H and R′″ is methyl.

17. A compound according to either claim 1 or 2 wherein R² is selected from the group consisting of: Ar—C₀₋₆alkyl, R⁹C(O)—, R⁹SO₂, R⁹R¹¹NC(O)—, and

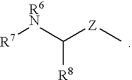

18. A compound according to claim 17 wherein R² is selected from the group consisting of: Ar—C₀₋₆alkyl, R⁹C(O)—, and R⁹SO₂.

19. A compound according to claim 18 wherein R² is R⁹SO₂.

20. A compound according to claim 17 wherein R⁶ is H.

21. A compound according to claim 17 wherein R⁷ is R¹⁰OC(O).

22. A compound according to claim 17 wherein R⁸ is C₁₋₆alkyl.

23. A compound according to claim 22 wherein R⁸ is isobutyl.

24. A compound according to claim 17 wherein R⁹ is selected from the group consisting of: C₁₋₆alkyl, Ar—C₀₋₆alkyl, —Ar—COOH and Het-C₀₋₆alkyl.

25. A compound according to claim 24 wherein:
C₁₋₆alkyl is selected from the group consisting of:
methyl;
ethyl, C₃₋₆cycloalkyl-C₀₋₆alkyl-substituted ethyl;
propyl;
butyl, C₁₋₆alkyl-substituted butyl;
tert-butyl; and
isopentyl;
Ar—C₀₋₆alkyl is selected from the group consisting of:
phenyl, halogen substituted phenyl, C₁₋₆alkoxy phenyl, C₁₋₆alkyl substituted phenyl, cyanophenyl, C₁₋₆alkyl sulfonyl substituted phenyl;
toluyl, Het-substituted toluyl; and
naphthylenyl;
—Ar—COOH is benzoic acid;
Het-C₀₋₆alkyl is selected from the group consisting of:
benzo[1,3]dioxolyl;
benzo[1,2,5]oxadiazolyl;
pyridinyl, 1-oxy-pyridinyl, C₁₋₆alkyl pyridinyl;
thiophene;
thiazolyl;
1H-imidazolyl, C₁₋₆alkyl substituted imidazolyl;
1H-[1,2,4]triazolyl, C₁₋₆alkyl substituted 1H-[1,2,4]triazolyl;
isoxazolyl, and C₁₋₆alkyl substituted isoxazolyl.

26. A compound according to claim 25 wherein:
ethyl is 2-cyclohexyl-ethyl;
butyl is 3-methylbutyl;
phenyl is selected from the group consisting of: 3,4-dichlorophenyl, 4-bromophenyl, 2-fluorophenyl, 3-fluorophenyl 4-fluorophenyl, 2-chlorophenyl, 3-chlorophenyl, 4-chlorophenyl, 3-methoxyphenyl, 4-methoxyphenyl, 3,4-dimethoxyphenyl, 2-cyanophenyl; 4-ethyl-phenyl, 2-methyl phenyl, 4-methyl phenyl, 4-methanesulfonyl phenyl, 2-methanesulfonyl phenyl; and
naphthylen-2-yl;
benzoic acid is 2-benzoic acid;
benzo[1,3]dioxolyl is benzo[1,3]dioxol-5-yl;
benzo[1,2,5]oxadiazolyl is benzo[1,2,5]oxadiazol-4-yl;
pyridinyl is selected from the group consisting of: pyridin-2-yl, pyridin-3-yl, 3-methyl-pyridin-2-yl, and 6-methyl-pyridin-2-yl;
1-oxy-pyridinyl is selected from the group consisting of: 1-oxy-pyridin-2-yl and 1-oxy-pyridin-3-yl;
thiopheneyl is thiophene-2-yl;
thiazolyl is thiazol-2-yl;
1H-imidazolyl is selected from the group consisting of: 1H-imidazol-2-yl, 1H-imidazol-4-yl, 1-methyl-1H-imidazol-2-yl, 1-methyl-1H-imidazolyl, and 1,2-dimethyl-1H-imidazol-4-yl;
1H-[1,2,4]triazolyl is selected from the group consisting of: 1H-[1,2,4]triazol-3-yl and 5-methyl-1H-[1,2,4]triazol-3-yl; and
3,5-dimethyl-isoxazolyl is 3,5-dimethyl-isoxazol-4-yl.

27. A compound according to either claim 1 or 2 wherein:
R² is selected from the group consisting of:
Ar—C₀₋₆alkyl, R⁹C(O)—, R⁹SO₂, R⁹R¹¹NC(O)—, and 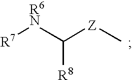
R⁴ is selected from the group consisting of: R⁵OC(O)—, R⁵C(O)— or R⁵SO₂—;
R⁵ is selected from the group consisting of: C₁₋₆alkyl, C₂₋₆alkenyl, C₃₋₆cycloalkyl-C₀₋₆alkyl, C₂₋₆alkanonyl, Ar—C₀₋₆alkyl and Het-C₀₋₆alkyl;
R⁶ is H;
R⁷ is R¹⁰OC(O);
R⁸ is C₁₋₆alkyl;
R⁹ is selected from the group consisting of: C₁₋₆alkyl, Ar—C₀₋₆alkyl, —Ar—COOH and Het-C₀₋₆alkyl;
R¹⁰ is selected from the group consisting of: C₁₋₆alkyl, Ar—C₀₋₆alkyl and Het-C₀₋₆alkyl;
R′ is H;
R″ is H; and
R′″ is methyl.

28. A compound according to claim 27 wherein:
R² is selected from the group consisting of: Ar—C₀₋₆alkyl, R⁹C(O)— and R⁹SO₂;
R⁴ is R⁵C(O)—; and
in R⁵:
C₁₋₆alkyl is selected from the group consisting of:
methyl, halogenated methyl, C₁₋₆alkoxy substituted methyl, heterocycle substituted methyl;
ethyl, heterocycle substituted ethyl;
butyl, aryl substituted butyl; and
isopentyl;
C₃₋₆cycloalkyl-C₀₋₆alkyl is cyclohexyl;
C₂₋₆alkenyl is selected from the group consisting of:
butenyl, and aryl substituted butenyl;
C₂₋₆alkanonyl is selected from the group consisting of:
acetyl; and
pentanonyl;
Ar—C₀₋₆alkyl is selected from the group consisting of:
phenyl, phenyl substituted with one or more halogens, phenyl substituted with one or more aryloxy or C₁₋₆alkoxy groups, phenyl substituted with one or more C₁₋₆alkyl sulfonyl groups;
benzyl; and
naphthylenyl; and
Het-C₀₋₆alkyl is selected from the group consisting of:
benzo[1,3]dioxolyl;
furanyl, nitro substituted furanyl, halogen substituted furanyl, aryl substituted furanyl, C₁₋₆alkyl substituted furanyl;
tetrahydrofuranyl;
benzofuranyl, C₁₋₆alkoxy substituted benzofuranyl, halogen substituted benzofuranyl, C₁₋₆alkyl substituted benzofuranyl;
napththo[2,1-b]-furanyl, C₁₋₆alkyl substituted napththo[2,1-b]-furanyl;
benzo[b]thiophenyl, C₁₋₆alkoxy substituted benzo[b]thiophenyl;
quinolinyl;
quinoxalinyl;
1,8 naphthyridinyl;
indolyl, C₁₋₆alkyl substituted indolyl;
pyridinyl, C₁₋₆alkyl substituted pyridinyl, 1-oxy-pyridinyl;
furo[3,2-b]-pyridinyl, C₁₋₆alkyl substituted furo[3,2-b]-pyridinyl;
thiophenyl, C₁₋₆alkyl substituted thiophenyl, halogen substituted thiophenyl;
thieno[3,2-b]thiophenyl C₁₋₆alkyl substituted thieno[3,2-b]thiophen-2-yl;
isoxazolyl, C₁₋₆alkyl substituted isoxazolyl;
oxazolyl, aryl substituted oxazolyl, C₁₋₆alkyl substituted oxazolyl; and
1H-benzoimidazolyl.

29. A compound according to claim 28 wherein:
halogenated methyl is trifluoromethyl;
C₁₋₆alkoxy substituted methyl is selected from the group consisting of: phenoxy-methyl and 4-fluoro-phenoxy-methyl;
heterocycle substituted methyl is 2-thiophenyl-methyl;
heterocycle substituted ethyl is piperidin-1-yl-ethyl;
aryl substituted butyl is 4-(4-methoxy)phenyl-butyl;
pentanonyl is 4-pentanonyl;
aryl substituted butenyl is 4,4-bis(4-methoxyphenyl)-but-3-enyl;
phenyl substituted with one or more halogens is selected from the group consisting of: 3,4-dichlorophenyl and 4-fluorophenyl;
phenyl substituted with one or more aryloxy or C₁₋₆alkoxy groups is selected from the group consisting of: 3,4-dimethoxy-phenyl and 3-benzyloxy-4-methoxy-phenyl;
phenyl substituted with one or more C₁₋₆alkyl sulfonyl groups is 4-methanesulfonyl-phenyl;
naphthylenyl is naphthylen-2-yl;
benzo[1,3]dioxolyl is benzo[1,3]dioxol-5-yl,
furanyl is furan-2-yl;
nitro substituted furanyl is 5-nitro-furan-2-yl;
aryl substituted furanyl is selected from the group consisting of: 5-(4-nitrophenyl)-furan-2-yl, 5-(3-triflouromethyl-phenyl)-furan-2-yl, and 5-(4-chloro-phenyl)-furan-2-yl);
halogen substituted furanyl is 5-bromo-furan-2-yl;
C₁₋₆alkyl substituted furanyl is selected from the group consisting of: 3-methyl-furan-2-yl, 4-methyl-furan-2-yl, 2,5-dimethyl-furan-2-yl, and 2,4-dimethyl-furan-2-yl;
tetrahydrofuranyl is tetrahydrofuran-2-yl;
benzofuranyl is benzofuran-2-yl;
C₁₋₆alkoxy substituted benzofuranyl is selected from the group consisting of: 5-(2-piperazinfcarboxylic acid tert-butyl ester-ethoxy) benzofuran-2-yl, 5-(2-morpholino-4-yl-ethoxy)-benzofuran-2-yl, 5-(2-piperazin-1-yl-ethoxy)benzofuran-2-yl, 5-(2-cyclohexyl-ethoxy)-benzofuran-2-yl, 7-methoxy-benzofuran-2-yl, 5-methoxy-benzofura-2-yl, 5,6-dimethoxy-benzofuran-2-yl5-methoxy-3-methyl-benzofuran-2-yl, 4-methoxy-3-methyl-benzofuran-2-yl, and 6-methoxy-3-methyl-benzofuran-2-yl;
halogen substituted benzofuranyl is selected from the group consisting of: 5-fluoro-benzofuran-2-yl 5,6-difluoro-benzofuran-2-yl5-fluoro-3-methyl-benzofuran-2-yl, and 6-fluoro-3-methyl-benzofuran-2-yl;
C₁₋₆alkyl substituted benzofuranyl is selected from the group consisting of: 3-methyl-benzofuran-2-yl, 3,5-dimethyl-benzofaran-2-yl, and 3-ethyl-benzofuran-2-yl;
napththo[2,1-b]-furanyl is napththo[2,1-b]-furan-2-yl;
C₁₋₆alkyl substituted napththo[2,1-b]-furanyl is 1-methyl-naphtho[2,1-b]-furan-2-yl;
benzo[b]thiophenyl is benzo[b]thiophen-2-yl;
C₁₋₆alkoxy substituted benzo[b]thiophenyl is 5,6-dimethoxy-benzo[b]thiophen-2-yl;
quinolinyl is selected from the group consisting of: quinolin-2-yl, quinolin-3-yl, quinolin-4-yl, quinolin-6-yl, and quinolin-8-yl;
quinoxalinyl is quinoxalin-2-yl;
1,8 naphthyridinyl is 1,8 naphthyridin-2-yl;
indolyl is selected from the group consisting of: indol-3-yl and indol-5-yl;
C₁₋₆alkyl substituted indolyl is N-methyl-indol-2-yl;
pyridinyl is selected from the group consisting of: pyridin-2-yl, pyridin-3-yl, and pyridin-5-yl;
1-oxy-pyridinyl is selected from the group consisting of: 1-oxy-pyridin-2-yl and 1-oxy-pyridin-3-yl;
C₁₋₆alkyl substituted pyridinyl is 2-methyl-pyridin-5-yl;
furo[3,2-b]-pyridinyl is furo[3,2-b]-pyridin-2-yl;
C₁₋₆alkyl substituted furo[3,2-b]-pyridinyl is 3-methyl-furo[3,2-b]-pyridin-2-yl;
thiophenyl is thiophen-3-yl;
halogen substituted thiophenyl is 4,5-dibromo-thiophen-2-yl;
C₁₋₆alkyl substituted thiophenyl is 5-methyl-thiophen-2-yl;
thieno[3,2-b]thiophenyl is thieno[3,2-b]thiophene-2-yl;
C₁₋₆alkyl substituted thieno[3,2-b]thiophen-2-yl is 5-tert-butyl-3-methyl thieno[3,2-b]thiophen-2-yl;
isoxazolyl is isoxazol-4-yl;
C₁₋₆alkyl substituted isoxazolyl is 3,5-dimethyl-isoxazol-4-yl;
oxazolyl is oxazol-4-yl;
aryl substituted oxazolyl is 5-methyl-2-phenyl oxazol-4-yl;
C₁₋₆alkyl substituted oxazolyl is 2-phenyl-5-trifluoromethyl-oxazol-4-yl; and
1H-benzoimidazolyl is 1H-benzoimidazol-5-yl.

30. A compound according to either claim 28 or 29 wherein in R⁹:
C₁₋₆alkyl is selected from the group consisting of:
methyl;
ethyl, C₃₋₆cycloalkyl-C₀₋₆alkyl-substituted ethyl;
propyl;
butyl, C₁₋₆alkyl-substituted butyl;
tert-butyl; and
isopentyl;
Ar—C₀₋₆alkyl is selected from the group consisting of:
phenyl, halogen substituted phenyl, C₁₋₆alkoxy phenyl, C₁₋₆alkyl substituted phenyl, cyanophenyl, C₁₋₆alkyl sulfonyl substituted phenyl;
toluyl, Het-substituted toluyl; and
naphthylenyl;
—Ar—COOH is benzoic acid;
Het-C₀₋₆alkyl is selected from the group consisting of:
benzo[1,3]dioxolyl;
benzo[1,2,5]oxadiazolyl;
pyridinyl, 1-oxy-pyridinyl, C₁₋₆alkyl pyridinyl;
thiopheneyl;
thiazolyl;
1H-imidazolyl, C₁₋₆alkyl substituted imidazolyl;
1H-[1,2,4]triazolyl, C₁₋₆alkyl substituted
1H-[1,2,4]triazolyl;
isoxazolyl, and C₁₋₆alkyl substituted isoxazolyl.

31. A compound according to claim 30 wherein:
ethyl is 2-cyclohexyl-ethyl;
butyl is 3-methylbutyl;
phenyl is selected from the group consisting of: 3,4-dichlorophenyl, 4-bromophenyl, 2-fluorophenyl, 3-fluorophenyl 4-fluorophenyl, 2-chlorophenyl, 3-chlorophenyl, 4-chlorophenyl, 3-methoxyphenyl, 4-methoxyphenyl, 3,4-dimethoxyphenyl, 2-cyanophenyl; 4-ethyl-phenyl, 2-methyl phenyl, 4-methyl phenyl, 4-methanesulfonyl phenyl, 2-methanesulfonyl phenyl; and
naphthylen-2-yl;
benzoic acid is 2-benzoic acid;
benzo[1,3]dioxolyl is benzo[1,3]dioxol-5-yl;
benzo[1,2,5]oxadiazolyl is benzo[1,2,5]oxadiazol-4-yl;
pyridinyl is selected from the group consisting of: pyridin-2-yl, pyridin-3-yl, 3-methyl-pyridin-2-yl, and 6-methyl-pyridin-2-yl;
1-oxy-pyridinyl is selected from the group consisting of: 1-oxy-pyridin-2-yl and 1-oxy-pyridin-3-yl;
thiopheneyl is thiophene-2-yl;
thiazolyl is thiazol-2-yl;
1H-imidazolyl is selected from the group consisting of: 1H-imidazol-2-yl, 1H-imidazol-4-yl, 1-methyl-1H-imidazol-2-yl, 1-methyl-1H-imidazol-4-yl, and 1,2-dimethyl-1H-imidazol-4-yl;
1H-[1,2,4]triazolyl is selected from the group consisting of: 1H-[1,2,4]triazol-3-yl and 5-methyl-1H-[1,2,4]triazol-3-yl; and
3,5-dimethyl-isoxazolyl is 3,5-dimethyl-isoxazol-4-yl.

32. A compound according to claim 27 wherein:
R² is R⁹SO₂;
R⁴ is R⁵C(O);
R⁵ is selected from the group consisting of: benzofuran-2-yl, 3-methyl-benzofuran-2-yl, 5-methoxybenzofuran-2-yl, thieno[3,2-b]thiophene-2-yl, quinoxalin-2-yl, and quinolin-2-yl, and
R⁹ is selected from the group consisting of: pyridin-2-yl and 1-oxy-pyridin-2-yl.

33. A compound according to claim 32 wherein R⁵ is selected from the group consisting of: benzofuran-2-yl and thieno[3,2-b]thiophene-2-yl.

34. A compound according to claim 33 wherein R⁵ is benzofuran-2-yl.

35. A compound according to claim 32 wherein R⁹ is pyridin-2-yl.

36. A compound according to either claim 1 or 2-selected from the group consisting of:
benzofuran-2-carboxylic acid {1-[(S)-3-oxo-1-(pyridine-2-sulfonyl)-azepan-4-ylcarbamoyl]-cyclohexyl}-amide;
benzofuran-2-carboxylic acid {1-[(R)-3-oxo-1-(pyridine-2-sulfonyl)-azepan-4-ylcarbamoyl]-cyclohexyl}-amide;
thieno[3,2-b]thiophene-2-carboxylic acid {1-[(+/−)-3-oxo-1-(pyridine-2-sulfonyl)-azepan-4-ylcarbamoyl]-cyclohexyl}-amide;
benzofuran-2-carboxylic acid {1-[(4S,7R)-7-methyl-3-oxo-1-(pyridine-2-sulfonyl)-azepan-4-ylcarbamoyl]-cyclohexyl}-amide;
thieno[3,2-b]thiophene-2-carboxylic acid {1-[(4S,7R)-7-methyl-3-oxo-1-(pyridine-2-sulfonyl)-azepan-4-ylcarbamoyl]-cyclohexyl}-amide;
2,2,4-trideutero-benzofuran-2-carboxylic acid {1-[(S)-3-oxo-1-(pyridine-2-sulfonyl)-azepan-4-ylcarbamoyl]-cyclohexyl}-amide;
2,2,4-trideutero-benzofuran-2-carboxylic acid {1-[(4S,7R)-7-methyl-3-oxo-1-(pyridine-2-sulfonyl)-azepan-4-ylcarbamoyl]-cyclohexyl}-amide;
2,2,4-trideutero-thieno[3,2-b]thiophene-2-carboxylic acid {1-[(S)-3-oxo-1-(pyridine-2-sulfonyl)-azepan-4-ylcarbamoyl]-cyclohexyl}-amide; and
2,2,4-trideutero-thieno[3,2-b]thiophene-2-carboxylic acid {1-[(4S,7R)-7-methyl-3-oxo-1-(pyridine-2-sulfonyl)-azepan-4-ylcarbamoyl]-cyclohexyl}-amide.

37. A pharmaceutical composition comprising a compound according to any one of claims 1 to 36 and a pharmaceutically acceptable carrier, diluent or excipient.

38. A method of inhibiting a protease, comprising administering to a patient in need thereof an effective amount of a compound according to any one of claims 1 to 36.

39. A method according to claim 38 wherein said protease is selected from the group consisting of a cysteine protease and a serine protease.

40. A method according to claim 39 wherein said protease is a cysteine protease.

41. A method according to claim 40 wherein said cysteine protease is cathepsin K.

42. A method of treating a disease characterized by bone loss comprising inhibiting said bone loss by administering to a patient in need thereof an effective amount of a compound according to any one of claims 1 to 36.

43. A method according to claim 42 wherein said disease is osteoporosis.

44. A method according to claim 42 wherein said disease is periodontitis.

45. A method according to claim 42 wherein said disease is gingivitis.

46. A method of treating a disease characterized by excessive cartilage or matrix degradation comprising inhibiting said excessive cartilage or matrix degradation by administering to a patient in need thereof an effective amount of a compound according to claims 1 to 36.

47. A method according to claim 46 wherein said disease is osteoarthritis.

48. A method according to claim 46 wherein said disease is rheumatoid arthritis.

49. A compound of Formula II: 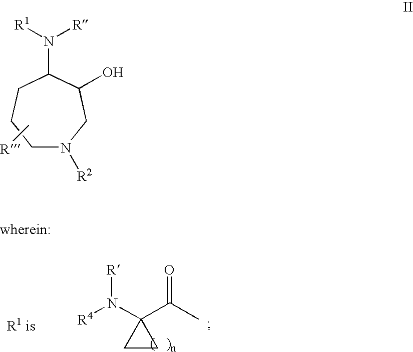
R² is selected from the group consisting of: H, C₁₋₆alkyl, C₃₋₆cycloalkyl-C₀₋₆alkyl, Ar—C₀₋₆alkyl, Het-C₀₋₆alkyl, R⁹C(O)—, R⁹C(S)—, R⁹SO₂—, R⁹OC(O)—, R⁹R¹¹NC(O)—, R⁹R¹¹NC(S)—, R⁹(R¹¹)NSO₂— 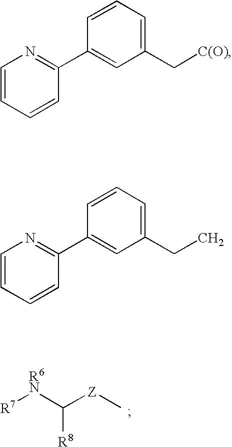
and R⁹SO₂R¹¹NC(O)—;
R⁴ is selected from the group consisting of: H, Cl₆alkyl, C₃₋₆cycloalkyl-C₀₋₆alkyl, Ar—C₀₋₆alkyl, Het-C₀₋₆alkyl, R⁵C(O)—, R⁵C(S)—, R⁵SO₂—, R⁵OC(O)—, R⁵R¹²NC(O)—, and R⁵R¹²NC(S)—;
R⁵ is selected from the group consisting of: H, C₁₋₆alkyl, C₂₋₆alkenyl, C₂₋₆alkynyl, C₃₋₆cycloalkyl-C₀₋₆alkyl, C₂₋₆alkanonyl, Ar—C₀₋₆alkyl and Het-C₀₋₆alkyl;
R⁶ is selected from the group consisting of: H, C₁₋₆alkyl, Ar—C₀₋₆alkyl, or Het-C₀₋₆alkyl;
R⁷ is selected from the group consisting of: H, C₁₋₆alkyl, C₃₋₆cycloalkyl-C₀₋₆alkyl, Ar—C₀₋₆alkyl, Het-C₀₋₆alkyl, R¹⁰C(O)—, R¹⁰C(S)—, R¹⁰SO₂—, R¹⁰OC(O)—, R¹⁰R¹³NC(O)—, and R¹⁰R¹³NC(S)—;
R⁸ is selected from the group consisting of: H, C₁₋₆alkyl, C₂₋₆alkenyl, C₂₋₆alkynyl, HetC₀₋₆alkyl and ArC₀₋₆alkyl;
R⁹ is selected from the group consisting of: C₁₋₆alkyl, C₃₋₆cycloalkyl-C₀₋₆alkyl, Ar—C₀₋₆alkyl, —ArCOOH, and Het-C₀₋₆alkyl;
R¹⁰ is independently selected from the group consisting of: C₁₋₆alkyl, C₃₋₆cycloalkyl-C₀₋₆alkyl, Ar—C₀₋₆alkyl and Het-C₀₋₆alkyl;
R¹¹ is selected from the group consisting of: H, C₁₋₆alkyl, Ar—C₀₋₆alkyl, and Het-C₀₋₆alkyl;
R¹² is selected from the group consisting of: H, C₁₋₆alkyl, Ar—C₀₋₆alkyl, and Het-C₀₋₆alkyl;
R¹³ is selected from the group consisting of: H, C₁₋₆alkyl, Ar—C₀₋₆alkyl, and Het-C₀₋₆alkyl;
R′ is selected from the group consisting of: H, C₁₋₆alkyl, Ar—C₀₋₆alkyl, and Het-C₀₋₆alkyl;
R″ is selected from the group consisting of: H, C₁₋₆alkyl, Ar—C₀₋₆alkyl, or Het-C₀₋₆alkyl;
R′″ is selected from the group consisting of: H, C₁₋₆alkyl, C₃₋₆cycloalkyl-C₀₋₆alkyl, Ar—C₀₋₆alkyl, and Het-C₀₋₆alkyl;
Z is selected from the group consisting of: C(O) and CH₂;
n is an integer of from 1 to 5;
and pharmaceutically acceptable salts, hydrates and solvates thereof.

50. A compound according to claim 49 selected from the group consisting of:
benzofuran-2-carboxylic acid {1-[(+/−)-3-hydroxy-1-(pyridine-2-sulfonyl)-azepan-4-ylcarbamoyl]-cyclohexyl}-amide;
thieno[3,2-b]thiophene-2-carboxylic acid {1-[(+/−)-3-hydroxy-1-(pyridine-2-sulfonyl)-azepan-4-ylcarbamoyl]-cyclohexyl}-amide;
benzofuran-2-carboxylic acid {1-[(3S,4S,7R)-3-hydroxy-7-methyl-1-(pyridine-2-sulfonyl)-azepan-4-ylcarbamoyl]-cyclohexyl}-amide; and
thieno[3,2-b]thiophene-2-carboxylic acid {1-[(3S,4S,7R)-3-hydroxy-7-methyl-1-(pyridine-2-sulfonyl)-azepan-4-ylcarbamoyl]-cyclohexyl}-amide.

51. A process for the synthesis of a compound according to claim 1 comprising the step of oxidizing a corresponding compound of claim 49 with an oxidant to provide the compound of Formula (I) as a mixture of diastereomers.

52. The process of claim 51 wherein the oxidant is sulfur trioxide pyridine complex in DMSO and triethylamine.

53. The process of claim 51 further comprising the step of separating the diasteromers by separating means.

54. The process of claim 53 wherein said separating means is high presssure liquid chromatography (HPLC).

55. The process of claim 51 further comprising the step of deuterating said diastereomers with a deuterating agent.

56. The process of claim 55 wherein said deuterating agent is CD₃OD: D₂O (10:1) in triethylamine.

57. Use of a compound according to any one of claims 1 to 36 in the manufacture of a medicament for use in inhibiting a protease selected from the group consisting of a cysteine protease and a serine protease.

58. A use according to claim 57 wherein said protease is a cysteine protease.

59. A use according to claim 58 wherein said cysteine protease is cathepsin K.

60. Use of a compound according to any one of claims 1 to 36 in the manufacture of a medicament for use in treating a disease characterized by bone loss.

61. A use according to claim 60 wherein said disease is osteoporosis.

62. A use according to claim 60 wherein said disease is periodontitis.

63. A use according to claim 60 wherein said disease is gingivitis.

64. Use of a compound according to any one of claims 1 to 36 in the manufacture of a medicament for use in treating a disease characterized by excessive cartilage or matrix degradation.

65. A use according to claim 64 wherein said disease is osteoarthritis.

66. A use according to claim 64 wherein said disease is rheumatoid arthritis.
